(19) United States
(12) Patent Application Publication
(10) Pub. No.: US 2025/0326867 A1
(43) Pub. Date: Oct. 23, 2025

(54) De novo designed chlorophyll special pair proteins

(71) Applicant: University of Washington, Seattle, WA (US)

(72) Inventors: David Baker, Seattle, WA (US); Nathan Ennist, Seattle, WA (US); Shunzhi Wang, Seattle, WA (US); Derrick Hicks, Seattle, WA (US); Adam Moyer, Seattle, WA (US)

(21) Appl. No.: 19/180,644

(22) Filed: Apr. 16, 2025

Related U.S. Application Data
(60) Provisional application No. 63/636,001, filed on Apr. 18, 2024.

(51) Int. Cl. C07K 19/00; C07K 14/00; C07K 17/00; C12N 15/62

(52) U.S. Cl. CPC C07K 19/00; C07K 14/00; C07K 17/00; C12N 15/62; C07K 2319/00

(57) ABSTRACT

Polypeptides are provide having an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-17, wherein the polypeptide binds to a chlorophyll (Chl) dimer, and scaffolds thereof.

### FEDERAL FUNDING STATEMENT

This invention was made with government support under Grant No. 2459-1671, awarded by the Advanced Research Projects Agency-Energy (ARPA-E). The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Apr. 15, 2025 having the file name “25-0264-US.xml” and is 83,089 bytes in size.

### BACKGROUND

Photosynthetic proteins manipulate the distances and angles between chlorophyll (Chl) molecules to tune excitonic coupling and control absorption and fluorescence spectra, excited state dynamics, energy transfer, and electron tunneling. This control enables light harvesting and charge separation with quantum yields of 97% or higher under favorable conditions. Natural photosynthesis can guide the development of synthetic biology for renewable fuel production, but only if we can determine the structure-function relationships required for efficient solar-to-fuel energy conversion and build new structures that exploit this knowledge. Chl special pairs have attracted great interest as primary electron donors, but the complexity of natural photosystems makes it difficult to study these Chls directly. Small molecule mimics of special pairs are labor-intensive to synthesize, overlook the role of protein matrix effects that are important in native special pairs, and lack the fine control over Chl-Chl distances and orientations needed to reproduce the precise geometries of native special pairs. No structures of Chl dimers in designed proteins have been determined experimentally. Systematic methods of assembling Chl dimers with predefined geometries are lacking, making it difficult to correlate structure and function. Despite decades of active research, there has been no generalizable strategy to assemble Chl dimers that precisely match special pair geometries.

### SUMMARY

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-17, wherein the polypeptide binds to a chlorophyll (Chl) dimer. In one embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-3 and 17. In a further embodiment, at least 1, 2, 3, 4, 5, 6, 7, 8, 9, 10, 11, 12, 13, 14, or all Chlorophyll (Chl)-contacting residues are identical, or conservatively substituted, relative to the reference sequence. In another embodiment, at least 1, 2, 3, 4, 5, 6, 7, 8, 9, 10, 11, 12, 13, 14, or all Chl-contacting residues are identical relative to the reference sequence. In one embodiment, at least 1, 2, 3, 4, 5, 6, 7, 8, 9, 10, or all identified protein-protein residues are identical (not substituted), or conservatively substituted, relative to the reference sequence. In another embodiment, at least 1, 2, 3, 4, 5, 6, 7, 8, 9, 10, 11, 12, 13, 14, or all identified protein-protein interface residues are identical, relative to the reference sequence. In a further embodiment, all Chl-contacting residues and all identified protein-protein are identical, relative to the reference sequence.

The disclosure also provides fusion proteins, comprising the polypeptide of any embodiment or combination of embodiments herein, and one or more functional domains at the N-terminus and/or at the C-terminus of the polypeptide.

The disclosure further provides nucleic acids encoding the polypeptide or fusion protein of any embodiment or combination of embodiments herein; expression vectors comprising the nucleic acid operatively linked to a suitable control element, including but not limited to a promoter; and host cells comprising the polypeptide, fusion protein, nucleic acid, and/or expression vector of any embodiment or combination of embodiments herein.

In one embodiment, the disclosure provides homodimers of the polypeptide of any embodiment or combination of embodiments herein. In a further embodiment, the homodimer comprises a Chl dimer bound to the homodimer.

The disclosure also provides scaffolds comprising homodimers of any embodiment or combination of embodiments herein. In one embodiment, the scaffold comprises
(a) a plurality of homodimers of a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:17; and
(b) a plurality of homotrimers of a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:18;
wherein the plurality of homodimers and the plurality of homotrimers non-covalently interact to form the scaffold. In another embodiment, the scaffold further comprises a plurality of Chl dimers bound to the plurality of homodimers.

The disclosure also provides compositions, comprising one or more polypeptide, fusion protein, homodimer, or scaffold of any embodiment or combination of embodiments herein, covalently linked to an electrode.

In another aspect, the disclosure provides methods comprising use of the scaffold of any embodiment or combination of embodiments herein for any suitable purpose, including but not limited to as a synthetic photosystem for new energy conversion technologies, photodynamic therapy, redox- or light-responsive biosensing, fluorescent reporters, optogenetics, light-gated enzymes, photoenzymes, photoprotection in biological systems, nitrogen fixation, carbon sequestration, enhanced crop yields for food or bioenergy production, or electrical-to-fuel energy transduction for energy storage.

### DESCRIPTION OF THE FIGURES

### Petition to Accept Color Drawings

The file of this patent contains at least one drawing executed in color. Copies of this patent with color drawing(s) will be provided by the Office upon request and payment of the necessary fee.

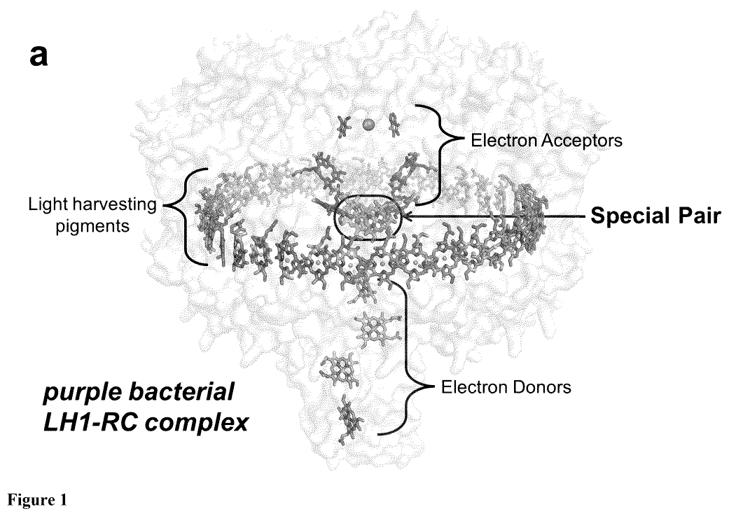

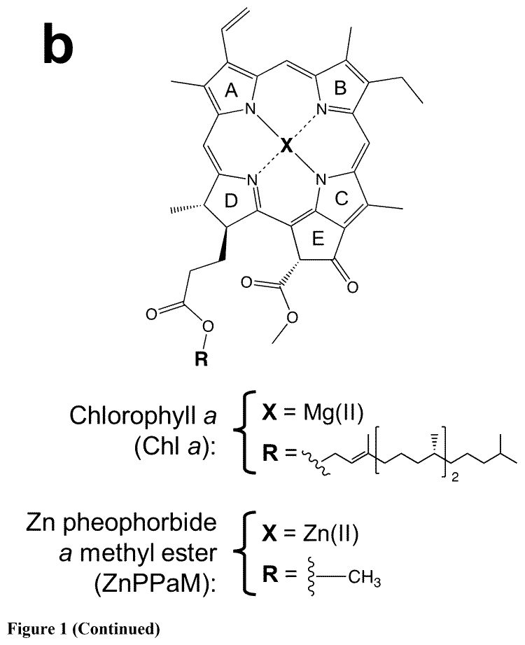

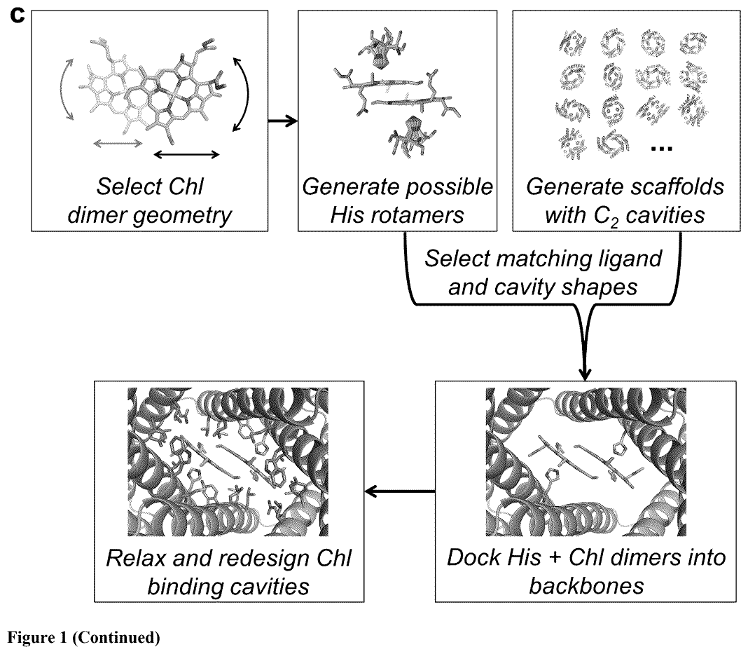

FIG. 1. Computational design of Chl special pair proteins. (a) Cryo-electron microscopic (cryo-EM) structure of native photosynthetic LH1-RC complex from purple non-sulfur bacteria (Blastochloris viridis) (PDB ID: 6ET5) (Qian et al. 2018). The special pair is shown in green and cyan, electron acceptors in red, electron donors in orange, and light-harvesting pigments in blue. Hydrocarbon tail groups and carotenoids have been removed for clarity. (b) Chemical structures of two chlorin compounds, Chl a and Zn pheophorbide a methyl ester (ZnPPaM). Chl a and ZnPPaM have similar spectroscopic properties. (c) Computational design of Chl special pair proteins begins with selection of a Chl dimer geometry and generation of inverse His rotamers. His-Chl dimers are docked into designed homodimers, and the Chl binding pockets are redesigned using Rosetta™ FastDesign.

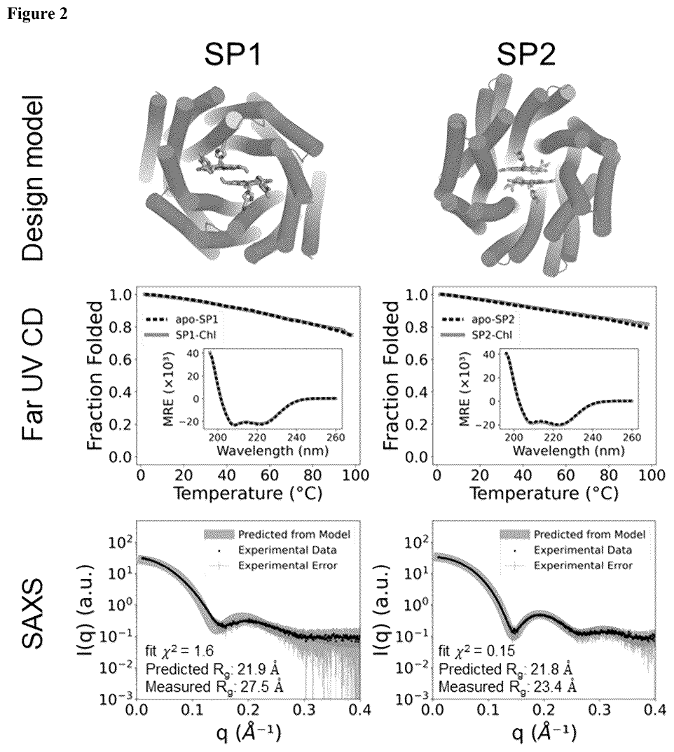

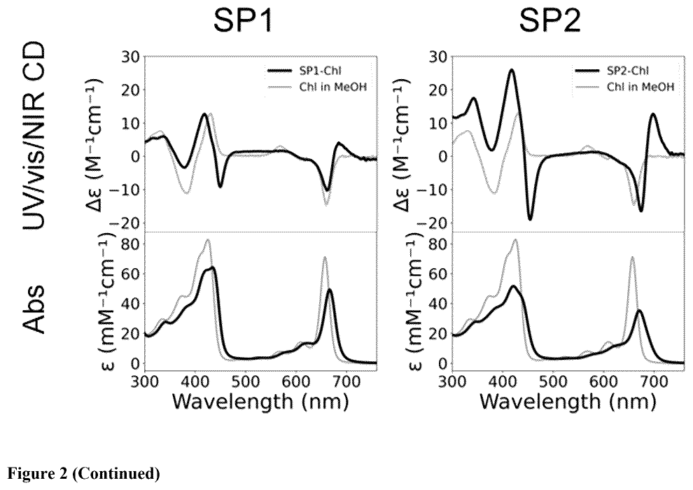

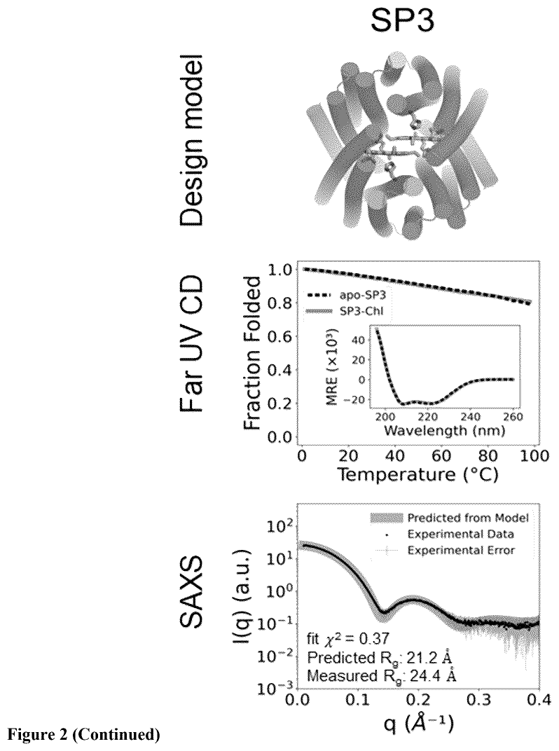

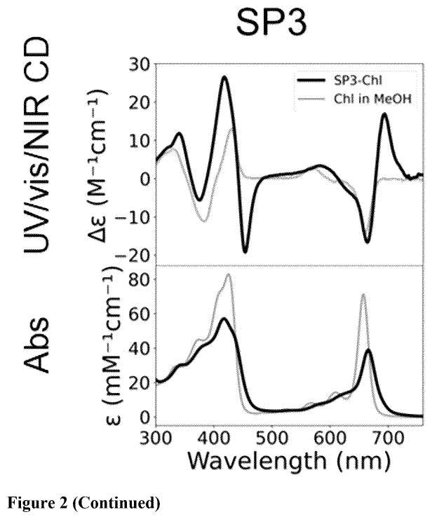

FIG. 2. Folding, stability and ZnPPaM binding of SP proteins. SP protein design models (top) are displayed with α-helices represented as cylinders and Chl molecules as sticks. Far UV CD signals at 222 nm monitored with increasing temperature show proteins to be highly thermostable in both apo-states (black dashed traces) and ZnPPaM-bound states (green traces). Far UV CD spectra measured at 25° C. (inset) have features typical of highly α-helical proteins including minima at 208 and 222 nm. Binding of ZnPPaM has little effect on secondary structure composition (green traces). Molar residue ellipticity (MRE) on the y-axis is given in deg cm² dmol⁻¹ residue⁻¹. Experimental Small Angle X-ray Scattering (SAXS) data, shown as black points with error bars in gray, are in good agreement with SAXS profiles predicted from apo-state design models using the FoXS server, shown as red traces (Schneidman-Duhovny et al. 2016, 2013). The UV/vis/NIR CD spectrum of each protein in the ZnPPaM-bound state is shown in comparison to a control spectrum of ZnPPaM in methanol (MeOH) with the absorbance (Abs) spectrum of the same sample beneath. In each case, the protein-bound dimer acquires a positive band at ˜690 nm, consistent with calculations based on dimer geometries (vide infra). Unbound ZnPPaM was removed by sterile filtration and PD-10 column chromatography prior to data collection.

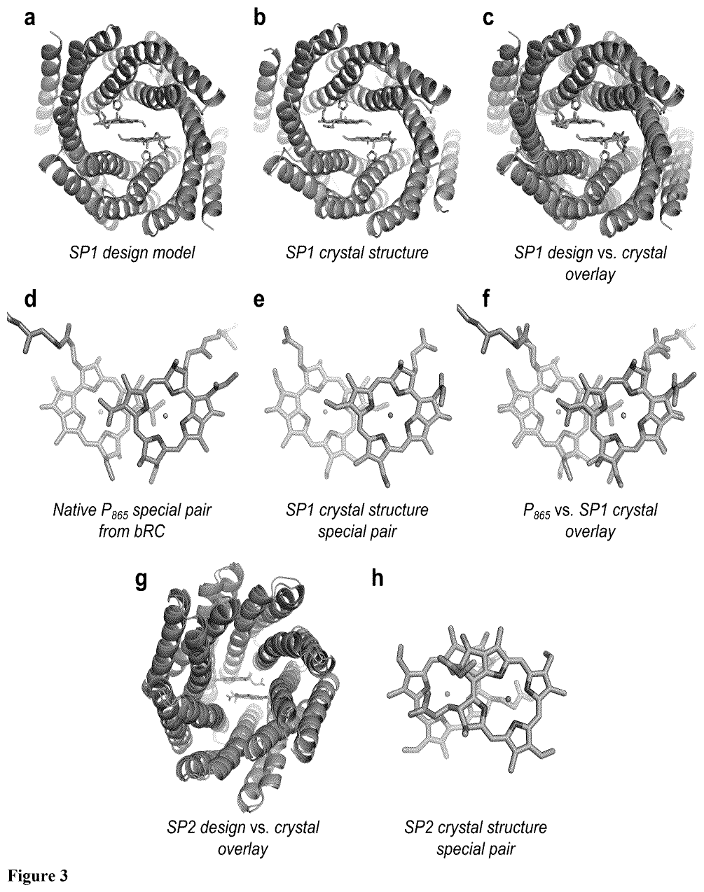

FIG. 3. X-ray crystal structures of designed SP proteins. (a) Rosetta™ design model of SP1. (b) SP1 crystal structure at 2.0 Å resolution with ZnPPaM molecules bound (PDB ID: 7UNJ). (c) SP1 Rosetta™ design model (gray) aligns to SP1 crystal structure (green) with 1.6 Å C_{α} atom RMSD. (d) The BChl a special pair from a 2.5 Å resolution cryo-EM structure of the purple bacterial RC-LH1 complex (Rhodobacter sphaeroides) (PDB ID: 7PIL) (Qian et al. 2021). (e) The ZnPPaM dimer from the SP1 crystal structure shown in panel (b). (f) The ZnPPaM dimer of the SP1 crystal structure (green) aligns with the native purple bacterial special pair (blue) to 0.23 Å RMSD across corresponding atoms of the tetrapyrrole rings. (g) Rosetta™ design model of SP2 (gray) aligns to the holo-state SP2 crystal structure (green, PDB ID: 7UNI) with 1.4 Å C_{α} atom RMSD. (h) The ZnPPaM dimer in the SP2 crystal structure (green) deviates from the predicted dimer geometry (not shown; 3.5 Å RMSD).

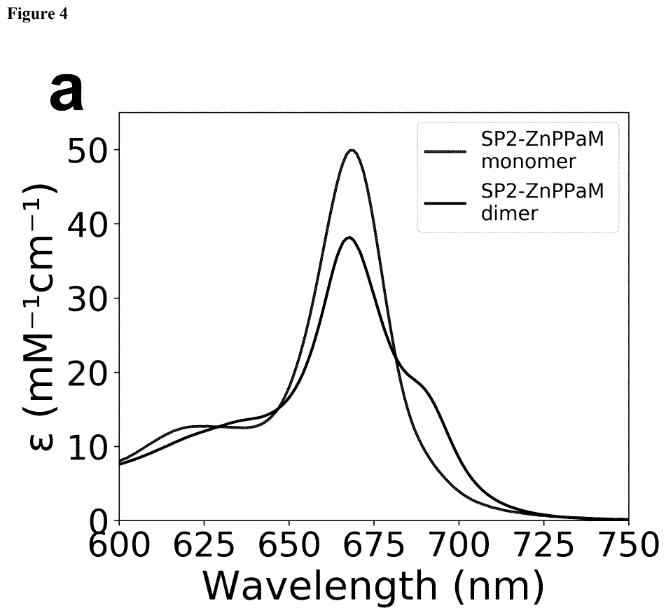

FIG. 4. Spectral shift on ZnPPaM dimer binding in SP2 protein. Molar absorptivity in solution of ZnPPaM when bound to SP2 protein as a monomer (blue trace) and as a dimer (black trace).

FIG. 5. SP2 functions as an energy transfer acceptor for native light harvesting protein. (Normalized absorbance (Abs) and fluorescence emission (Em) spectra of CpcA-PEB and SP2-ZnPPa. Spectra were collected in solution.

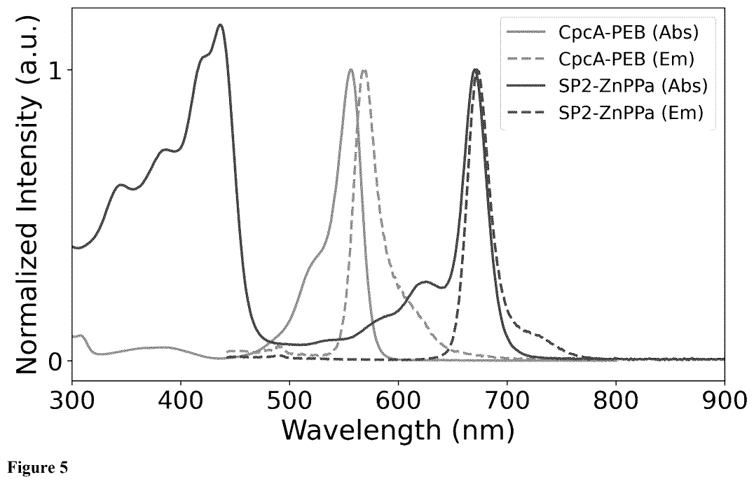

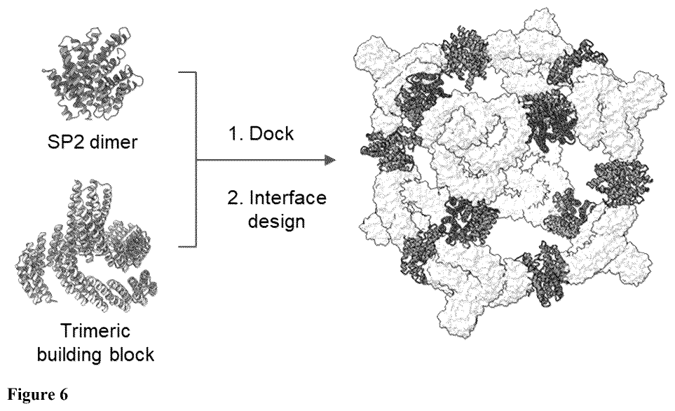

FIG. 6. Design of a nanocage that assembles Chl dimer proteins. Nanocage design model was generated by docking SP2 dimer with trimeric building block to form 2-component octahedral nanocage architecture. Rosetta™ sequence design was used to stabilize interfaces between dimeric and trimeric building blocks.

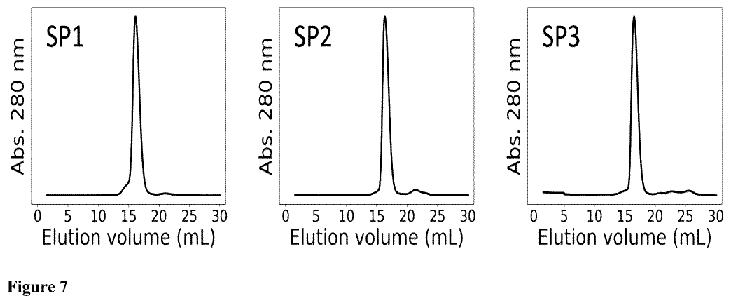

FIG. 7. Size exclusion chromatography (SEC) traces of purified SP apo-proteins. Proteins were injected as 0.5 mL aliquots onto a Superdex™ 200 Increase 10/300 GL column (Cytiva Life Sciences). The narrow, isolated peaks indicate that the samples contain monodisperse protein. Running buffer was 150 mM NaCl, 10 mM Tris at pH 8. Proteins were purified by Ni-NTA and one round of SEC prior to collecting the data shown.

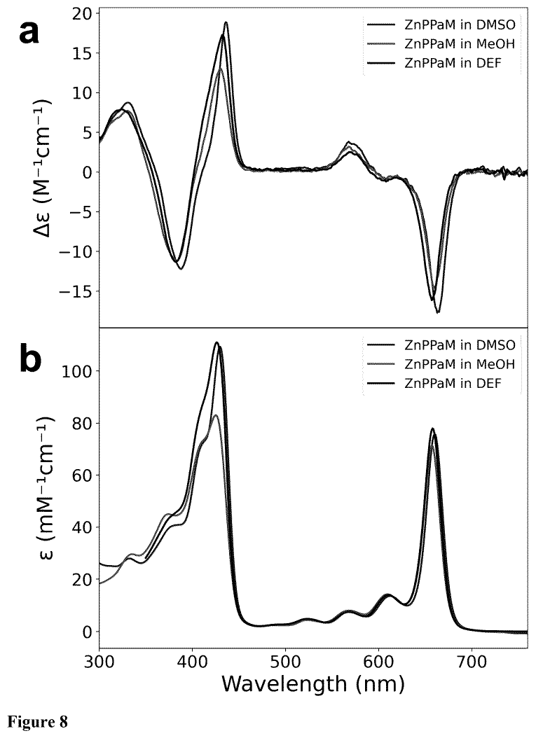

FIG. 8. CD and absorbance spectra of Zn pheophorbide a methyl ester (ZnPPaM) in organic solvents. (a) CD spectra of ZnPPaM (plotted as difference molar extinction coefficients, Δε) in dimethyl sulfoxide (DMSO), methanol (MeOH), and diethylformamide (DEF). Spectra were collected with 15 μM ZnPPaM in 1 cm cuvettes. Spectra of 1, 5, and 75 μM ZnPPaM in DEF (not shown) did not differ appreciably from the 15 μM ZnPPaM spectrum in DEF (black trace), meaning that there is no sign of concentration-dependent aggregation. All CD spectra were collected on a J-1500 CD spectrophotometer at 25° C. Each spectrum is the average of 10 scans using a 3 nm bandwidth, 50 nm/min scanning speed, 4 second data integration time, and a data interval of 1 nm. (b) Molar absorptivity (¿) of the same samples collected using a V-750 spectrophotometer with a 1 nm bandwidth, 400 nm/min scanning speed, 0.24 second ultraviolet/visible (UV/vis) response, and a data interval of 1 nm.

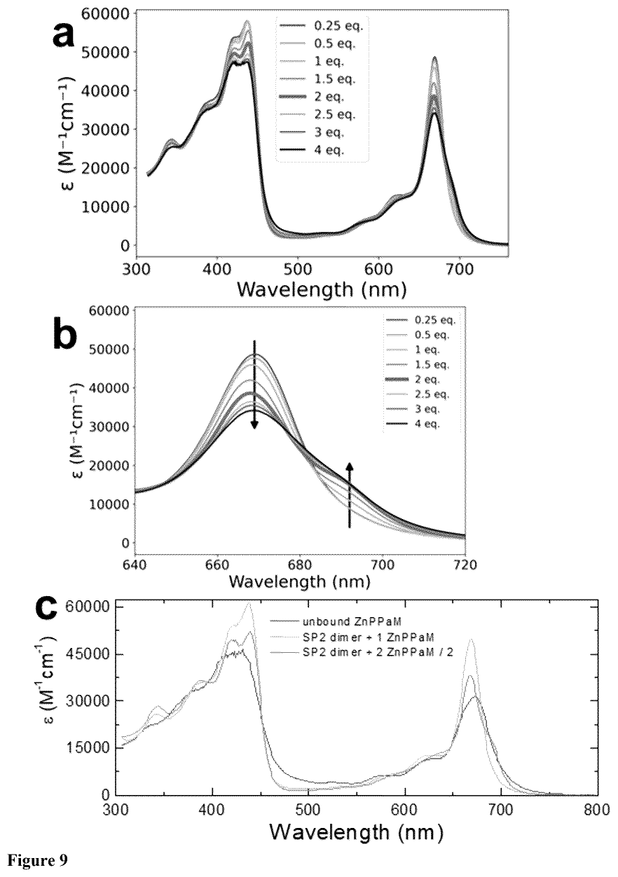

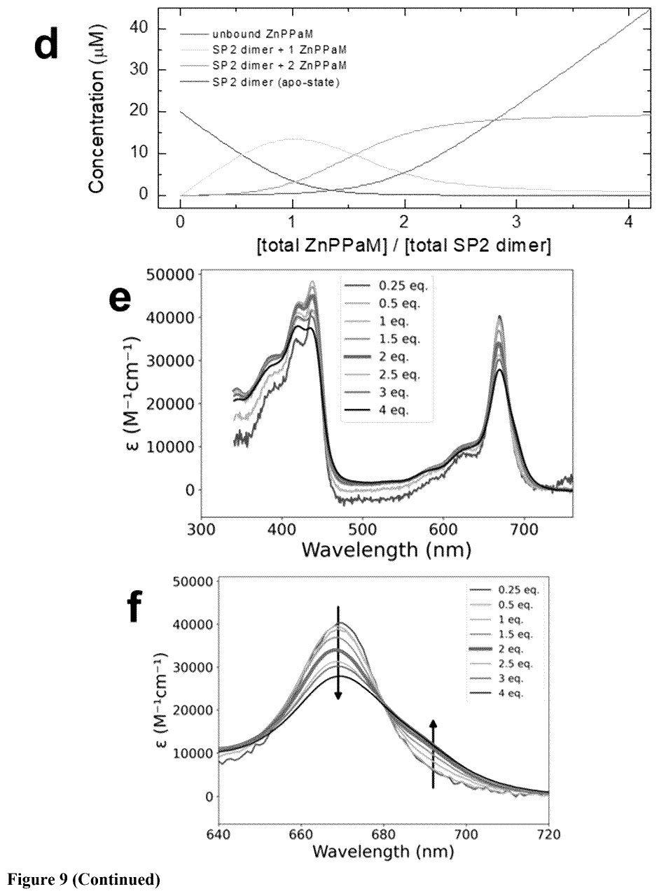

FIG. 9. SP2-ZnPPaM binding titration monitored by UV/vis absorbance. (a) UV/vis titration of ZnPPaM into a solution of 20 μM SP2 dimer (40 M of SP2 monomer) in 150 mM NaCl, and 10 mM Tris at pH 8. Samples were prepared separately with 0.25 to 4 equivalents (eq.) of ZnPPaM per SP2 protein dimer (2.0 eq. ZnPPaM would be a stoichiometric amount). The y-axis is the molar extinction coefficient, &, or OD divided by ZnPPaM concentration. ZnPPaM was added to protein samples from a DMSO stock solution. Samples were incubated overnight for 20 hours at 4° C. before measurement at room temperature. (b) The same data as in panel (a) zoomed in on the Q_{y} region. Arrows indicate the direction & changes as a function of increasing ZnPPaM concentration. (c) Fitted spectra and (d) species concentration vs. molar ratio of ligand to protein from fitting of the absorbance spectra in panels (a) and (b). During fitting, only the P+L→PL and PL+L→PL₂ reactions were considered, and ZnPPaM aggregation was disregarded. (P represents SP2 dimer protein and L represents ZnPPaM ligand). Curve fitting gives dissociation constants (K_{D}s) of 110 nM and 2.0 μM for K_{D1} and K_{D2}, respectively. Fittings were performed with an algorithm that combines singular value decomposition and least squares regression, as implemented in ReactLab™ Equilibria. (e) Full spectra and (f) the Q_{y} region of a second titration with SP2 dimer protein concentration held at 2.0 μM. At 2.0 μM protein, the intensities of the spectral features at 669 and 692 nm depend upon the molar ratio of protein to ZnPPaM in a manner similar to the 20 μM protein titration shown in panels (a) and (b).

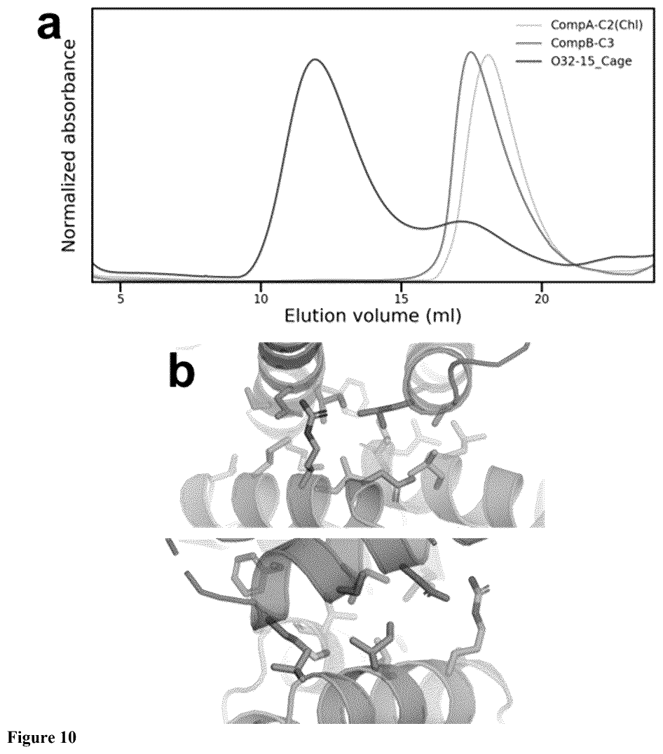

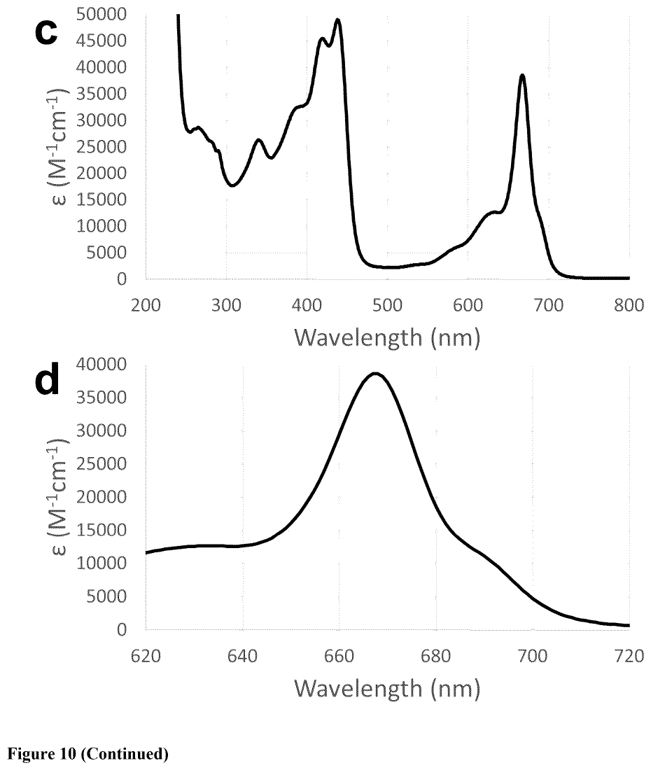

FIG. 10. Design and characterization of chlorophyll-binding O32-15 nanocage. (a) Size exclusion chromatography (SEC) chromatogram shows octahedral nanocage (green trace) elutes at the volume expected for the target 2-component cage (48-subunit) on a Superose™ 6 Increase 10/300 gel filtration column (Cytiva). The O32-15 cage was assembled in vitro by stoichiometric mixture of the individually produced and purified designed components: chlorophyll dimer (O32-15A, light green) and trimeric component (O32-15B, grey), respectively. (b) Two views of the design model of the cage interface within the two-component O32-15 chlorophyll binding cage. (c) UV/vis absorbance spectrum and (d) closeup of Q_{y} region of octahedral nanocage with chlorophyll bound after PD-10 column and sterile filtration. The spectrum includes a shoulder on the Q_{y} absorbance band at 692 nm, similar to SP2. The sample was prepared by adding a 1.8-fold excess of ZnPPaM (32 μM) to 2.5 mL of 17.6 μM nanocage (Chl binding site concentration) in 150 mM NaCl, 10 mM Tris at pH 8, and 0.3% w/v 3-((3-cholamidopropyl) dimethylammonio)-1-propanesulfonate (CHAPS) detergent. Sample was purified by PD-10 column to remove excess ZnPPaM and sterile filtered with a 0.22 μm syringe filter prior to collecting the absorbance spectrum shown.

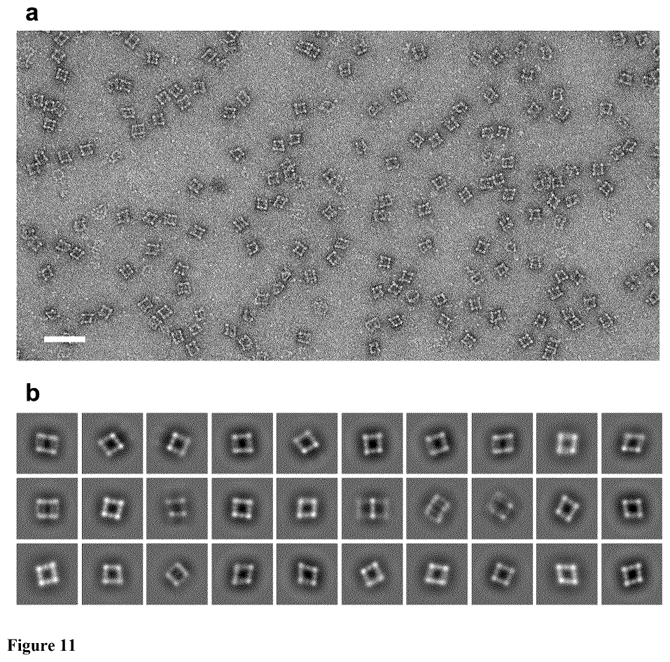

FIG. 11. Negative-stain electron microscopy of purified O32-15 nanocage. (a) Representative negative stain electron micrograph of O32-15 (scale bar at bottom left, 100 nm). (b) 2D class averages of negative stain electron microscopy data created in CryoSparc (Punjani et al. 2017).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), RosettaCommons.org, and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be deleted). In various embodiments, any 1, 2, 3, 4, or 5 N-terminal or C-terminal amino acids of the polypeptides of the disclosure may be deleted relative to the reference sequence.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-17, wherein the polypeptide binds to a chlorophyll (Chl) dimer.

The primary electron donor in photosynthetic charge separation is a chlorophyll “special pair,” a pigment that accepts excitation energy from the light-harvesting complex and initiates an electron transfer cascade in the reaction center. A special pair is a chlorophyll dimer with offset π-stacking interactions in which the precise inter-chromophore distance and orientation are thought to determine the photophysical and functional properties. As disclosed in the examples that follow, the polypeptides of the disclosure are de novo designed chlorophyll (Chl) special pair proteins, that are capable of homodimerization and binding of a Chl pair. The polypeptides can be used, for example, as energy transfer acceptors when paired with native light-harvesting proteins, and to form scaffolds to serve as a de novo photosynthetic chromatophore-like structure.

The amino acid sequences of SEQ ID NO:1-17 are provided in Tables 1 and 2

| | | Amino acid sequence (after | |
|---|---|---|---|
| | | TEV protease digestion). | Interface residues |
| SEQ | | Residues in parentheses are | (Underlined and bold-font |
| ID | SP | optional, and may be | residues are preferably |
| NO | design | present or may be absent | conserved) |
| 1 | SP1 | (GG)DSRELIARYQILLAELAAIRADI | Chl contacting residues : 18, |
| | | AAERTGDPYVRKLARELKRLAQEAAEE | Y11, Q12, L15, M64. L67, |
| | | VKRDPSSSDVNMALLLILLMIELAVRA | L68, L71, L75, W119, L120, |
| | | LEAAERTGDPEVRELAAELVWLAVEAA | H123, M126, L127, and W130 |
| | | EEVQRNPSSSDVWLALHLIMLAIWAAV | Protein-protein interface |
| | | AALEAAERTGDPEVRELARELVRLAVE | residues : L7, Y11, L13, L18, |
| | | AAEEVQRNPSSKEVYMALLLILIAILE | I21, A23, 125, M176, L178, |
| | | AVLSLLRAERSGDPEKREKARERVREA | I183, L186, and L193 |
| | | VERAEEVQR | |
| 2 | SP2 | (SGSGSS)DEEFKFLATEAKMLITAAE | Chl contacting residues : |
| | | RLAGTDP(E)LQEMVALIKKELEQAER | L27, M75, A79, I128, V132, |
| | | TFRNGDKSEAQRQLEFVLTAARAVMNV | A135, H184, M188, A191, |
| | | AAAANAAGTDP(ELIEMVLRILKQ)LK | A240, and A243 |
| | | EAIRTFQN(GDQEE)AETQLRFVLRAA | Protein-protein interface |
| | | IAVAVVAAALVLAGTDPELQEMV(KQI | residues : E8, F12, T15, |
| | | LEELKQAIETFARGDK)EKALTQLLFV | M19, T22, A23, L27, A83, |
| | | AWAAHAVAMIAAAANLA(GTD)PRLQQ | E170, T174, L177, W181, |
| | | QVKEILEKLKEAIETFQKGDEEQAFRQ | F229, A232, A237, L241, and |
| | | LAEVLAEAALVALRAALTN | L244 |
| 3 | SP3 | (SGSS)EELARESAEAAWRLAQAGTRL | Chl contacting residues : |
| | | MLAWIRGDLKEIAEALIELARAVQELA | G22, L25, M26, W29, L44, |
| | | RVAKEYGNDELAKTAALLAAHVAMLAI | L69, L70, H73, M76, L77, |
| | | LVLIRAIKEGDDEVRELAKTAIKLAST | L80, A151, L154, L155, L158, |
| | | AAKIVLDALPTAEEVRQITLLAKLAEE | F198, and L202 |
| | | AADKKNEDSALAVGIAAAAVLLALLAL | Protein-protein interface |
| | | EAAQKAGIEEAEKGARLLLKLAMDAAR | residues: A8, A12, A15, W16, |
| | | KKNPEEALAVFNAALDVSIALKLLQSA | L18, A19, Q20, T23, M26, |
| | | KRAGSEETRKLAEEMLRQALERARK | L27, I30, L63, I148, L195, |
| | | | F198, N199, L202, I201, |
| | | | L210, L225, and M229 |
| 4 | tj79C2- | (SGSGS)RYDELNARLLILLAELAAER | |
| | H62A | ADIAAERTGDPRVRELARELKRLAQEA | |
| | | AEEVKRDPSSWSVLHALWLIVVAIEAA | |
| | | VRALEAAERTGDPEVRELARELVRLAV | |
| | | EAAEEVQRNPSSWDVEMALLLIVFAIE | |
| | | AAVRALEAAERTGDPEVRELARELVRL | |
| | | AVEAAEEVQRNPSSKEVDMALLLILIA | |
| | | ILEAVLSLLRAERSGDPEKREKARERV | |
| | | REAVERAEEVQRDPSGW | |
| 5 | tj79C2- | (SGSGS)REDELLARYLILLAELAAEE | |
| | H65A | ADIAAERTGDPRVRELARELKRLAQEA | |
| | | AEEVKRDPSSSDVNHALHLIVLAILIA | |
| | | VEALEAAERTGDPEVRELARELVRLAV | |
| | | EAAEEVQRNPSSSDVLKALILILLAII | |
| | | QAVKALEAAERTGDPEVRELARELVRL | |
| | | AVEAAEEVQRNPSSKEVTMALLLILIA | |
| | | ILEAVLSLLRAERSGDPEKREKARERV | |
| | | REAVERAEEVQRDPSGW | |
| 6 | tj79C2- | (SGSGS)REDELFARYLILLAELAAER | |
| | H65B | ADIAAERTGDPRVRELARELKRLAQEA | |
| | | AEEVKRDPSSSDVNLALHLIVLAILAA | |
| | | VEALEAAERTGDPEVRELARELVRLAV | |
| | | EAAEEVQRNPSSSDVLKALMLIVLAIL | |
| | | QAVQALEAAERTGDPEVRELARELVRL | |
| | | AVEAAEEVQRNPSSKEVYMALLLILIA | |
| | | ILEAVLSLLRAERSGDPEKREKARERV | |
| | | REAVERAEEVQRDPSGW | |
| 7 | tj79C2- | (SGSGS)REDELIARYLILLAELAAER | |
| | H65C | ADIAAERTGDPRVRELARELKRLAQEA | |
| | | AEEVKRDPSSSDVFMALHLIVLAIQIA | |
| | | VKALEAAERTGDPEVRELARELVRLAV | |
| | | EAAEEVQRNPSSSDVFLALLLIVTAIM | |
| | | LAVEALEAAERTGDPEVRELARELVRL | |
| | | AVEAAEEVQRNPSSKEVLMALLLILIA | |
| | | ILEAVLSLLRAERSGDPEKREKARERV | |
| | | REAVERAEEVQRDPSGW | |
| 8 | tj79C2- | (SGSGS)REDELQARLLILLIELAAER | |
| | H65D | ADIAAERTGDPRVRELARELKRLAQEA | |
| | | AEEVKRDPSSSDVWLALHLIVIAILIA | |
| | | VEALEAAERTGDPEVRELARELVRLAV | |
| | | EAAEEVQRNPSSSDVLKALMLILIAIL | |
| | | LLLEALEAAERTGDPEVRELARELVRL | |
| | | AVEAAEEVQRNPSSKEVQMALLLILIA | |
| | | ILEAILSLLRAERSGDPEKREKARERV | |
| | | REAVERAEEVQRDPSGW | |
| 9 | tj79C2- | (SGSGS)REDELWARLLILLIELAAER | |
| | H65E | ADIAAERTGDPRVRELARELKRLAQEA | |
| | | AEEVKRDPSSSDVLLALHLIVIAILIA | |
| | | VEALEAAERTGDPEVRELARELVRLAV | |
| | | EAAEEVQRNPSSSDVLIALMLILIAIL | |
| | | LLLEALEAAERTGDPEVRELARELVRL | |
| | | AVEAAEEVQRNPSSKEVQMALLLILIA | |
| | | ILEAILSLLRAERSGDPEKREKARERV | |
| | | REAVERAEEVQRDPSGW | |
| 10 | tj79C2- | (SGSGS)REDELQARLQLLAAELAAEE | |
| | H69A | ADIAAERTGDPRVRELARELKRLAQEA | |
| | | AEEVKRDPSSSDVNAALVLITHAIMLA | |
| | | VEALKAAERTGDPEVRELARELVRLAV | |
| | | EAAEEVQRNPSSSDVNKALFLIMMAII | |
| | | TAVTALEAAERTGDPEVRELARELVRL | |
| | | AVEAAEEVQRNPSSKEVDMALLLILIA | |
| | | ILEAVLSLLRAERSGDPEKREKARERV | |
| | | REAVERAEEVQRDPSGW | |
| 11 | tj79C2- | (SGSGS)REDELHARYLILSAELAAER | |
| | H69B | ADIAAERTGDPRVRELARELKRLAQEA | |
| | | AEEVKRDPSSSDVNKALLLITHAILIA | |
| | | VAALEAAERTGDPEVRELARELVRLAV | |
| | | EAAEEVQRNPSSSDVNRALFLIMMAIM | |
| | | MAVKALEAAERTGDPEVRELARELVRL | |
| | | AVEAAEEVQRNPSSKEVDMALLLILIA | |
| | | ILEAVLSLLRAERSGDPEKREKARERV | |
| | | REAVERAEEVQRDPSGW | |
| 12 | tj79C2- | (SGSGS)REDELIARYQILLAELAAER | |
| | H121A | ADIAAERTGDPRVRELARELKRLAQEA | |
| | | AEEVKRDPSSSDVNMALLLILLMIELA | |
| | | VRALEAAERTGDPEVRELARELVRLAV | |
| | | EAAEEVQRNPSSSDVWQALHLIMLAIW | |
| | | AAVAALEAAERTGDPEVRELARELVRL | |
| | | AVEAAEEVQRNPSSKEVYMALLLILIA | |
| | | ILEAVLSLLRAERSGDPEKREKARERV | |
| | | REAVERAEEVQRDPSGW | |
| 13 | tj79C2- | (SGSGS)REDELLARLLLLLAELAAER | |
| | H121B | ADIAAERTGDPRVRELARELKRLAQEA | |
| | | AEEVKRDPSSSDVLMALLLIIMAIEAA | |
| | | VEALEAAERTGDPEVRELARELVRLAV | |
| | | EAAEEVQRNPSSSDVWWALHLILFAIQ | |
| | | LAVRALEAAERTGDPEVRELARELVRL | |
| | | AVEAAEEVQRNPSSKEVDMALLLILIA | |
| | | ILEAVLSLLRAERSGDPEKREKARERV | |
| | | REAVERAEEVQRDPSGW | |
| 14 | tj79C2- | (SGSGS)REDELLARLLILLAELAAER | |
| | H121C | ADIAAERTGDPRVRELARELKRLAQEA | |
| | | AEEVKRDPSSSDVLMLLLLIVLAIQAA | |
| | | VEALEAAERTGDPEVRELARELVRLAV | |
| | | EAAEEVQRNPSSSDVEMALHLILFAIQ | |
| | | LAVQALEAAERTGDPEVRELARELVRL | |
| | | AVEAAEELQRNPSSKEVQMALLLILIA | |
| | | ILEAVLSLLRAERSGDPEKREKARERV | |
| | | REAVERAEEVQRDPSGW | |
| 15 | tj79C2- | (SGSGS)REDELWARYTLLLAELAAER | |
| | H125A | ADIAAERTGDPRVRELARELKRLAQEA | |
| | | AEEVKRDPSSSDVYLALILIVMAIYMA | |
| | | VLALEAAERTGDPEVRELARELVRLAV | |
| | | EAAEEVQRNPSSSDVNKALLLIVHAIM | |
| | | AAVAALEAAERTGDPEVRELARELVRL | |
| | | AVEAAEEVQRNPSSKEVDMALLLIQIA | |
| | | ILEAVLSLLRAERSGDPEKREKARERV | |
| | | REAVERAEEVQRDPSGW | |
| 16 | tj79C2- | (SGSGS)REDELRARLLILLAELAAET | |
| | H132A | LDIIAERTGDPRVRELARELIRLLQEA | |
| | | AEEVKRDPSSSDVNEALKLIVEAIFMA | |
| | | IFILLFAEQTGDPEVRELARELVRLAV | |
| | | EAAEEVQRNPSSSDVNEALKLIIQAII | |
| | | LAVHALLAAEKTGDPEVRELARELVRL | |
| | | AVEAAEEVQRNPSSKEVDMALLLILIA | |
| | | ILEAVLSLLRAQRSGDPEKREKARERV | |
| | | REAVERAEEVQRDPSGW | |

Table 1. Amino acid sequences of designed chlorophyll special pair proteins. Shown are the protein sequences after expression using a pET-29b(+) vector and cleavage of N-terminal MGHHHHHHGSGSGENLYFQ (SEQ ID NO:19) sequence by TEV protease. Residues in parentheses are optional and may be present or may be deleted. Right-hand column: Chlorophyll-contacting residues and residues in the homodimer interface.

In one embodiment, the polypeptides comprise an amino acid sequence at 75% identical to the amino acid sequence selected from the group consisting of SEQ ID NO:1-17. In another embodiment, the polypeptides comprise an amino acid sequence at 85% identical to the amino acid sequence selected from the group consisting of SEQ ID NO:1-17. In a further embodiment, the polypeptides comprise an amino acid sequence at 95% identical to the amino acid sequence selected from the group consisting of SEQ ID NO:1-17. In one embodiment, the polypeptides comprise an amino acid sequence at 75% identical to the amino acid sequence selected from the group consisting of SEQ ID NO:1-3 and 17. In another embodiment, the polypeptides comprise an amino acid sequence at 85% identical to the amino acid sequence selected from the group consisting of SEQ ID NO:1-3 and 17. In a further embodiment, the polypeptides comprise an amino acid sequence at 95% identical to the amino acid sequence selected from the group consisting of SEQ ID NO:1-3 and 17.

In one embodiment, at least 1, 2, 3, 4, 5, 6, 7, 8, 9, 10, 11, 12, 13, 14, or all Chl-contacting residues are identical (not substituted), or conservatively substituted, relative to the reference sequence. The Chl-contacting residues for SEQ ID NO:1-3 are shown in the right-hand column of Table 1, and in Table 2 for SEQ ID NO:17. In one embodiment, at least 5 Chl-contacting residues are identical (not substituted), or conservatively substituted, relative to the reference sequence. In a further embodiment, at least 10 Chl-contacting residues are identical (not substituted), or conservatively substituted, relative to the reference sequence. In another embodiment, all Chl-contacting residues are identical (not substituted), or conservatively substituted, relative to the reference sequence.

As used herein, conservative amino acid substitutions involve replacing a residue by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe.

In a further embodiment, at least 1, 2, 3, 4, 5, 6, 7, 8, 9, 10, or all identified protein-protein residues are identical (not substituted), or conservatively substituted, relative to the reference sequence. The homodimer interface residues (and homodimer-homotrimer interface residues for SEQ ID NO:17) are shown the right-hand column of Table 1, and in Table 2 for SEQ ID NO:17. In one embodiment, at least 5 identified homodimer interface residues are identical (not substituted), or conservatively substituted, relative to the reference sequence. In a further embodiment, at least 10 identified homodimer interface residues are identical (not substituted), or conservatively substituted, relative to the reference sequence. In another embodiment, all identified homodimer interface residues are identical (not substituted), or conservatively substituted, relative to the reference sequence. In a further embodiment, all identified homodimer interface residues are identical (not substituted), relative to the reference sequence. In one embodiment, all Chl-contacting residues and all identified protein-protein are identical, relative to the reference sequence.

The disclosure also provides fusion proteins, comprising:
(a) the polypeptide of any embodiment or combination of embodiments disclosed herein; and
(b) one or more functional domains at the N-terminus and/or at the C-terminus of the polypeptide.

In these embodiments, any functional domain may be used. In various non-limiting embodiments, the functional domain may comprise, for example, a targeting domain, a detectable domain, a light-absorbing domain, and an electron-transfer domain.

In one embodiment, the one or more functional domain comprises a light-absorbing protein. Any light-absorbing protein may be incorporated as appropriate for an intended purpose. In some embodiments, the light-absorbing or electron-transfer protein is selected from the group consisting of rhodopsin, photopsin, melanopsin, light-harvesting complex (LHC) proteins (including but not limited to Lhcb1-3 in LHCII, Lhca1-4 in LHCI, and LH2 from purple non-sulfur bacteria); phycobiliproteins (including but not limited to including phycoerythrin, phycocyanin, and allophycocyanin); ferredoxin, flavodoxin, cytochrome complex, plastocyanin, fluorescent proteins (including but not limited to green fluorescent protein, red fluorescent protein, aequorin, luciferin, catalase, ELIP (early light-induced protein), Msf1 (light-harvesting complex-like protein involved in maintaining photosystem I and chlorophyll-binding proteins/complexes), and LHCR (Chl a-binding polypeptides associated with PSI, found in red algae.

In another aspect the disclosure provides nucleic acids encoding the polypeptide or fusion protein of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded peptide or chimeric molecular construct, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptide or fusion protein of the disclosure.

In a further aspect, the disclosure provides expression vectors comprising the nucleic acid of any aspect of the disclosure operatively linked to a suitable control sequence, such as a promoter. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in a host organism either as an episome or by integration into host chromosomal DNA. In various embodiments, the expression vector may comprise a plasmid, viral-based vector, or any other suitable expression vector.

In another aspect, the disclosure provides host cells that comprise the polypeptide, fusion protein nucleic acid and/or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In some embodiments, the cell comprises a cell that can be used for biomass production based on light absorption. Any such cells can be used, including but not limited to bacteria engineered to biosynthesize pigments like chlorophyll molecules, purple non-sulfur bacteria, cyanobacteria, or any cell with a chloroplast such as algae or plant cells. In these embodiments, the cells of the disclosure can absorb more of the spectrum from the sun (or artificial light source) resulting in more efficient biomass production.

In another embodiment, the disclosure provides homodimers of the polypeptide or fusion protein of any embodiment or combination of embodiments disclosed herein. As noted above, the polypeptides of the disclosure are capable of homo-dimerization and of binding to Chl-dimers. Thus, in one embodiment, the homodimers further comprise one or more Chl dimer bound to the homodimer.

In one embodiment, the disclosure provides scaffolds comprising a plurality of the homodimers of any embodiment or combination of embodiments disclosed herein. In this embodiment, the plurality of homodimers may be presented on any scaffold as appropriate, to permit display of the plurality of homodimers, and a plurality of any bound Chl pairs. In one embodiment, the plurality of homodimers comprise
(a) a plurality of homodimers of a polypeptide comprising an amino acid sequence at 75% identical to the amino acid sequence of SEQ ID NO:17; and
(b) a plurality of homotrimers of a polypeptide comprising an amino acid sequence at 75% identical to the amino acid sequence of SEQ ID NO:18;
wherein the plurality of homodimers and the plurality of homotrimers non-covalently interact to form the scaffold.

In this embodiment the polypeptide of SEQ ID NO:18 is capable of forming a homotrimer, which can non-covalently interact with the homodimer. The amino acid sequences of SEQ ID NO:17-18 are provided in Table 2. In one embodiment of any scaffold herein, the scaffold may further comprise a plurality of Chl dimers bound to the homodimers in the scaffold. In this embodiment, the scaffolds can be viewed as photosynthetic compartments analogous to thylakoids or chromatophores, and thus useful as synthetic photosystems for new energy conversion technologies. In some embodiments, the plurality of homodimer comprises 3, 4, 5 6, 7, 8, 9, 10, 11, 12, or more homodimers, each with a Chl dimer bound to it.

TABLE 2
Annotated amino acid sequences of designed nanocage proteins.
Underlined residues are those present in the trimer-dimer interface,
bold residues are present in the chlorophyll-binding pocket of the C2
dimer (sequence 17), and bold residues in the C3 trimer (sequence 18)
which were mutated relative to an earlier design (published in PMCID:
PMC5497568). The N-terminal Met residue and residues in the C-terminal
tags are italicized and in parentheses, and are optional and can be
present or deleted.

| SEQ ID | | |
|---|---|---|
| NO | Design | Amino acid sequence |
| 17 | C2 dimer | (M)DEEFKFLATEAKMLITAAERLAGTDPRLQEMVALIKKELEQAERTFRNGDK |
| | (based | SEAQRQLEFVLTAARAVMNVAAAANAAGTDPLLKAMVDAILWRLKEAIRTFQNG |
| | on SP2) | DEEAETQLRFVLRAAIAVAVVAAALVLAGTDPELQEMVEQIKDLLISAFMAGEK |
| | | ALTQLLEVAWAAHAVAMIAAAANLAGPRLQQQVKEILEKLKEAIETFQKGDEEQ |
| | | AFRQLAEVLAEAALVALRAALTN(LEHHHHHH) |
| 18 | C3 | (M)STKEKARQLAEEAKETAEKVGDPELIKLAEQASQEGDSEKAKAILLAAEAA |
| | trimer | RVAKEVGAPDLIRLARIAARVGASEAAKAILLAAEAARVAKEVGDPELERLALL |
| | | AAVLGDSEKAKAILLAAEAARVAKEVGDPELIKLALEAAERGDSEKAKAILLAA |
| | | EAARVAKEVGDPELIKLALEAARRGDSEKAKAILLAAEAARVAKEVGDPELIKL |
| | | ALEAARRGDSRKAEAILLAAEAARIAKEAGDPEARKKALEAARRGDRELATRIL |
| | | IEALLRLLKKSTAELKRATASLRAITEELKKNPSEDALVEHNRAIVEHNAIIVE |
| | | NNRIIAMVLEAIVRAI(GSWSGLEHHHHHH) |

In another embodiment, the disclosure provides compositions, comprising one or more polypeptide, fusion protein, homodimer, and/or scaffold covalently linked to an electrode. In one embodiment, the compositions further comprise light-harvesting and electron-transfer domains that participate in photon absorption and charge separation. In this embodiment, the compositions can be used, for example, to generate electrical power upon illumination of the composition.

In another aspect, the disclosure also provides method comprising use of the scaffold of any embodiment herein for any suitable purpose, including but not limited to as a synthetic photosystem for new energy conversion technologies, photodynamic therapy, redox- or light-responsive biosensing, fluorescent reporters, optogenetics, light-gated enzymes, photoenzymes, photoprotection in biological systems, nitrogen fixation, carbon sequestration, enhanced crop yields for food or bioenergy production, or electrical-to-fuel energy transduction for energy storage. See the examples for further details.

In another aspect, the disclosure provides polypeptides comprising an amino acid sequence at least 80%, 85%, 90%, 91%, 92%, 93%, 94%, 95%, 96%, 97%, 98%, 99%, or 100% identical to the amino acid sequence of SEQ ID NO:18, wherein all bold-font residues as shown in Table 2 are identical relative to the reference sequence. The polypeptides of this aspect are capable for forming homotrimers and can be used, for example, to generate scaffolds with compatible homodimers (including but not limited to homodimers of the polypeptide of SEQ ID NO: 17), via non-covalent interaction of homodimers and homotrimers in the scaffold. In a further embodiment, all underlined residues as shown in Table 2 for SEQ ID NO:18 are identical relative to the reference sequence,

### Examples

Abstract: Natural photosystems couple light harvesting to charge separation using a “special pair” of chlorophyll molecules that accepts excitation energy from the antenna and initiates an electron-transfer cascade. To investigate the photophysics of special pairs independent of complexities of native photosynthetic proteins, and as a first step towards synthetic photosystems for new energy conversion technologies, we designed C₂-symmetric proteins that precisely position chlorophyll dimers. X-ray crystallography shows that one designed protein binds two chlorophylls in a binding orientation matching native special pairs, while a second positions them in a previously unseen geometry. Spectroscopy reveals excitonic coupling, and fluorescence lifetime imaging demonstrates energy transfer. We designed special pair proteins to assemble into 24-chlorophyll octahedral nanocages; the design model and cryo-EM structure are nearly identical. The design accuracy and energy transfer function of these special pair proteins suggest that de novo design of artificial photosynthetic systems is within reach of current computational methods.

Photosynthetic proteins manipulate the distances and angles between chlorophyll (Chl) molecules to tune excitonic coupling and control absorption and fluorescence spectra, excited state dynamics, energy transfer, and electron tunneling. Natural photosynthesis can guide the development of synthetic biology for renewable fuel production, but only if we can determine the structure-function relationships required for efficient solar-to-fuel energy conversion and build new structures that exploit this knowledge. Chl special pairs have attracted great interest as primary electron donors, but the complexity of natural photosystems makes it difficult to study these Chls directly. Systematic methods of assembling Chl dimers with predefined geometries are lacking, making it difficult to correlate structure and function. Despite decades of active research, there has been no generalizable strategy to assemble Chl dimers that precisely match special pair geometries.

We sought the creation of stable, water-soluble proteins that assemble Chl dimers with predefined geometries and which can be built into extensive protein assemblies. Binding a small molecule as a dimer is a computational challenge, because the binding interface involves not just the protein but also the second small molecule, which has an independent set of rotational and translational degrees of freedom. To control these degrees of freedom, we sought to design homodimers with perfect two-fold cyclic (C₂) symmetry, which bind a C₂-symmetric Chl pair such that the C₂ symmetry axes of the protein and chromophore are coincident, similar to native reaction centers, which can have true C₂ symmetry or pseudo-C₂ symmetry (FIG. 1a). C₂ symmetry ensures that the two bound Chl molecules will have near-degenerate site energies, improving the resonance between pigment transitions necessary to create delocalized states. For Chl dimer protein scaffolds, we chose hyperstable C₂-symmetric repeat protein dimers containing symmetric pockets with tunable sizes and geometries. In this dimeric repeat protein architecture (FIG. 1c), the hydrophobic core is independent from the small molecule binding site, enabling full customization for binding with little impact on the overall protein structure. Several thousand C₂-symmetric homodimers that sample a wide range of superhelical curvature, rise, and radius parameters have been generated (Hicks et al. 2022; Fallas et al. 2017).

To probe the effect of geometry on Chl-Chl coupling, we set out to design a range of C₂-symmetric dimers that hold two closely interacting Chl molecules in varied geometries including the arrangement found in native special pairs. In native proteins, (B) Chls typically have a pentacoordinate central Mg(II) or Zn(II) ion with a histidine (His) Ne atom as the axial ligand. For each chosen special pair geometry, we built a His rotamer interaction field and stored the possible His-Chl interaction geometries in a hash table (FIG. 1c; see Methods for details). For each geometrically compatible C₂ scaffold, we cycled through His-Chl rotamers from the hash table, aligned them to the scaffold C₂-symmetry axis, and searched for matches of the His N—C_{α}-C backbone atoms to the backbone atoms of the residues lining the binding cavity. Scaffolds for which the His N—C_{α}-C backbone atoms aligned with corresponding atoms in the protein backbone, and which could accommodate the Chl dimer without clashes, were redesigned using symmetric Rosetta™ FastDesign to optimize hydrophobic packing and hydrogen bonding around the Chls (Maguire et al. 2021) (FIG. 1c). Designs were filtered based on the Rosetta™ full-atom energy, the solvent-accessible surface area of the Chl dimer (DSasa), His rotamers, and His Ne-metal ligation geometry. We selected 43 designs based on 13 unique scaffolds for experimental characterization (see Table 3 for amino acid sequences). We also characterized an additional 5 redesigned variants of one of the initial 43 designs after determination of its X-ray crystal structure provided clues to improve its function (vide infra). The protein monomer sizes range from 20.6 to 28.4 kDa (179 to 261 amino acids). We refer to these 48 designs as Chl Special Pair proteins, or SP for brevity.

Following SP protein expression in E. coli, SDS-PAGE gels showed that all 48 designs were present in the soluble fractions of lysates. Proteins were purified by Ni-NTA and size-exclusion chromatography (SEC) (FIG. 7). All SEC traces exhibited protein absorption at the elution volume expected for homodimer formation. Of 20 designs investigated by Small Angle X-ray Scattering (SAXS) in the apo-state, 15 had SAXS profiles suggesting a 3-dimensional shape consistent with the design model (FIG. 2, and Table 4) (Schneidman-Duhovny et al. 2016, 2013). A slightly lower predicted radius of gyration (R_{g}) value compared to experimental SAXS data is likely due to a dense hydration shell around the highly charged SP proteins (Svergun et al. 1998; Kim et al. 2016). The far ultraviolet (UV) circular dichroism (CD) spectra of three SP proteins that expressed in high yield (≥140 mg/L) show the proteins are highly α-helical with and without the synthetic Chl a derivative, Zn pheophorbide a methyl ester (ZnPPaM). Thermal denaturation curves monitored by the CD signal at 222 nm indicate that all three proteins are highly thermostable in the apo- and holo-states (FIG. 2).

At longer wavelengths in the UV/visible/near-infrared (UV/vis/NIR), CD spectra can serve as a convenient probe of excitonic interactions between Chls. Monomeric Chls including Chl a and ZnPPaM exhibit asymmetric negative CD signals in the Q_{y} region near ˜670 nm (FIG. 8) (Lindorfer, Müh, and Renger 2017). When Chl dimers are arranged in chiral protein environments, however, excitonic interactions can produce delocalized transitions with chiral character, yielding CD signals that are stronger and conservative (i.e., composed of a bisignate doublet that integrates to zero). FIG. 2 shows that ZnPPaM bound to the SP1, SP2, and SP3 proteins have bisignate CD features in the Q_{y} region (in the red part of spectrum), consistent with excitonic coupling between the Chls. As shown in Table 5, the Q_{y} CD features of SP2 and SP3 are substantially stronger relative to their Q_{y} absorption bands than is the Q_{y} CD signal of monomeric ZnPPaM in organic solvent. ZnPPaM binding titrations of SP2 and SP3 monitored by CD in the Q_{y} region show that the CD doublets are attributable to the binding of ZnPPaM dimers. Curve fitting of the CD titrations yields SP2-ZnPPaM dissociation constants (K_{D}s) of 300 nM for K_{D1} and 2.5 μM for K_{D2}, and SP3-ZnPPaM K_{D}s of 800 nM for K_{D1} and 1.0 μM for K_{D2} (data not shown). Due to the lower CD signal of the SP1-ZnPPaM complex, we instead used absorbance and fluorescence to measure the SP1-ZnPPaM interaction. Absorption titrations yielded curve fits with K_{D1} and K_{D2} values of 290 nM and 430 nM for SP1, 110 nM and 2.0 μM for SP2, and 350 nM and 940 nM for SP3, respectively (FIG. 9). Fluorescence titrations analyzed using a 1:1 binding model of protein monomer to ZnPPaM yield K_{D} estimates of 660 nM for SP1, 480 nM for SP2, and 120 nM for SP3; these K_{D} values approximate the average of K_{D1} and K_{D2} for each protein.

Based on the results of SEC, SAXS, and spectroscopic experiments (FIG. 2 and FIGS. 7-9), we selected promising candidates for X-ray crystallographic structure determination. We solved the crystal structures of SP1 and SP2, and found that both had protein backbone conformations that matched the corresponding design models to within 1.7 Å C_{α} RMSD (FIG. 3).

The X-ray crystal structure of SP1 was solved in the ZnPPaM-bound state to 2.0 Å resolution, revealing a special pair geometry closely matching that of purple photosynthetic bacteria (FIG. 3a-f). The rotameric state of the Zn-ligating His121 is identical to that in the design model, and several hydrophobic and T-stacking interactions form as designed. Hydrogen bonds to the ring E ketone group, shown to be important for modulating special pair redox potentials (Lin et al. 1994), form with Gln10 in both ZnPPaM molecules, in agreement with the design model. Alignment of the tetrapyrrole rings of the SP1-ZnPPaM dimer to nine native BChl a special pairs from different species of purple bacteria gave RMSDs of 0.23-0.28 Å (Cao et al. 2022; Niwa et al. 2014; Qian et al. 2021, 2022; Selikhanov et al. 2020; Swainsbury et al. 2021; Tani et al. 2020; Yu et al. 2018). (See details of RMSD measurements in Methods). For comparison, the special pairs of two crystal structures of the same Thermochromatium tepidum LH1-RC complex deviate from one another by 0.22 Å RMSD across the tetrapyrrole rings (PDB IDs: 3WMM and 5Y5S) (Niwa et al. 2014; Yu et al. 2018). The RMSD between the ZnPPaM dimer in the SP1 crystal structure and its design model is 0.25 Å.

SP2 was intended to assemble a ZnPPaM dimer with a conformation significantly different from native special pairs in order to investigate the effect of dimer geometry. The SP2 crystal structure was solved in both the apo-state and the ZnPPaM-bound state to 2.4 and 2.5 Å resolution, respectively. The apo- and holo-state amino acid backbones both agree with the SP2 design model to within 1.4 Å RMSD (FIG. 3g). The holo-state crystal structure has two copies of the SP2 dimer in the asymmetric unit; alignment of the two ZnPPaM dimers shows their binding geometries are equivalent, with an RMSD of 0.22 Å over the tetrapyrrole rings. The ZnPPaM molecules are ligated by His178 as in the SP2 design model. After alignment of the crystal structure and design model protein backbones, the corresponding tetrapyrrole rings are approximately coplanar. Despite the accuracy of the protein backbone design, the crystal structure shows the ZnPPaM molecules are rotated and translated relative to the design model (3.5 Å RMSD across tetrapyrrole ring atoms). Compared to the apo-state crystal structure, the SP2 binding cavity widens by ˜1.6 Å in the presence of ZnPPaM; this expansion provides the extra volume needed for the ZnPPaM molecules to adopt their unexpected conformation. While the ZnPPaM dimer in SP2 differs from the design model, the crystal structure nevertheless satisfies the objective of creating a non-native dimer geometry.

The 3.05 Å-resolution apo-state structure of design SP3x, which shares 94% sequence identity with SP3, was solved, and the SP3x homodimeric design model agreed with the X-ray crystal structure to 1.61 Å C_{α} RMSD (data not shown).

The absorption and fluorescence spectra of native special pairs are shifted compared to monomeric (B) Chls, in part due to excitonic coupling between the (B) Chls, which enables them to act as exciton traps (Taylor and Kassal 2019; Gorka et al. 2021; van Amerongen, Valkunas, and van Grondelle 2000; Swainsbury et al. 2023). The SP2-ZnPPaM dimer absorbance spectrum presents a red-shifted shoulder in solution. Analysis of SP2-ZnPPaM absorbance binding titrations (FIG. 4a and FIG. 9) shows that whereas the Q_{y} transition of monomeric ZnPPaM in SP2 has an absorbance maximum at 669 nm with an extinction coefficient (ε_{669 nm}) of 49,900 M⁻¹ cm⁻¹, the SP2-ZnPPaM dimer spectrum has its Q_{y} maximum slightly shifted to 668 nm with a decreased ε_{668 nm} of 38,200 M⁻¹ cm⁻¹. While the monomer has no discernable spectroscopic feature at 690 nm (its ε_{690 nm} is 9,400 M⁻¹ cm⁻¹), the SP2-ZnPPaM dimer spectrum has a distinct shoulder with ε_{690 nm} of 17,700 M⁻¹ cm⁻¹.

To investigate the origin of the SP2-ZnPPaM bands at 668 and 690 nm, and rule out the possibility that they represent two populations of distinct ZnPPaM oligomers, we collected low-temperature absorption and fluorescence spectra (FIG. 4b,c see Methods for details). We prepared a dimer sample with 2.0 molar equivalents of ZnPPaM per SP2 protein dimer and a monomer sample with only 0.3 molar equivalents per protein dimer, both in sucrose/trehalose films at 75 K. The monomer sample lacked a red-shifted shoulder (FIG. 4b), but in the SP2-ZnPPaM dimer sample, two emission bands were observed at 673 and 692 nm (FIG. 4c). Using the SP2-ZnPPaM dimer crystal structure (FIG. 3h), we obtained an excitonic coupling energy of 241 cm⁻¹ from time-dependent density functional theory calculations (see Methods for details). By comparison, the experimental SP2-ZnPPaM absorption features at 668 and 690 nm correspond to a Q_{y} peak splitting of 477 cm⁻¹ (a coupling of 239 cm⁻¹), consistent with the calculated value. We also simulated absorption and fluorescence spectra in the PigmentHunter™ application (Ahad, Lin, and Reppert 2023) (dashed lines in FIG. 4b,c; see Methods for details). The simulated dimer spectra succeeded in reproducing the weak oscillator strength of the lower-energy state and the large shift between absorption and fluorescence maxima. The close agreement between calculated and experimental data supports the conclusion that the SP2-ZnPPaM dimer system exhibits excitonic delocalization.

Calculations on the SP1-ZnPPaM dimer crystal structure (FIG. 3e) yielded a lower excitonic coupling of 87 cm⁻¹. Experimental low-temperature spectra of SP1-ZnPPaM in 40% glycerol showed only a modest broadening of the SP1-ZnPPaM dimer fluorescence emission band compared to the monomer, consistent with weaker excitonic coupling in SP1 (data not shown).

Further confirmation of excitonic coupling comes from comparing experimental and calculated CD spectra based on the ZnPPaM dimer geometries of design models and crystal structures. We found that the signs of the CD Cotton effects predicted from the crystal structures of SP1 and SP2 are consistent with the experimental signs in the Q_{y} region. In the SP3 dimer, the calculated spectrum based on the design model agrees with the experimentally-measured signs of the CD Cotton effects, suggesting that the P700-like ZnPPaM dimer in SP3 assembles as designed. (See Methods and FIGS. 10-11 for details of CD spectral calculations and simulations of Qₓ and Soret bands).

Native special pairs play a critical role as energy transfer acceptors for antenna proteins. To test whether our designed SP proteins participate in energy transfer with natural light-harvesting proteins, we analyzed excitation energy transfer in 2D surface arrays using Fluorescence Lifetime Imaging Microscopy (FLIM) in combination with nanoimprint lithography (Huang, Vasilev, and Hunter 2020) (FIG. 5). We selected the cyanobacterial antenna protein CpcA with attached phycoerythrobilin (PEB) as the energy transfer donor. CpcA-PEB was purified from E. coli, and gives a strong fluorescence emission maximum at 568 nm and emission extending past 630 nm (Barnett et al. 2017), overlapping with the excitation spectrum of Zn pheophorbide a (ZnPPa) when bound to SP2. Notably, Q_{y} peak splitting was not observed in SP2 assembled with ZnPPa instead of ZnPPaM (FIG. 5b), suggesting that the peripheral substituents of the chlorin play a significant role in excitonic coupling. To monitor energy transfer, ˜5 μm-wide linear arrays of CpcA-PEB and perpendicular linear arrays of SP2-ZnPPa were applied to a poly-L-lysine functionalized glass surface by contact printing (Huang, Vasilev, and Hunter 2020), creating intersection points where CpcA-PEB and SP2-ZnPPa interact and other locations in which only one of the proteins was present. Wide-field epifluorescence imaging with excitation at 450 nm was used to analyze the surface attachment; filtering emission at 620 nm preferentially displays regions of CpcA-PEB, while 680 nm preferentially shows SP2-ZnPPa. At the intersections between the lines, donor CpcA-PEB emission (620 nm filter) was decreased and SP2-ZnPPa acceptor (680 nm filter) was increased, indicating energy transfer from donor to acceptor (FIG. 5c).

To quantify the strength of the interaction between CpcA-PEB and SP2-ZnPPa, we used time-resolved single photon counting. The surface was illuminated with a 485 nm picosecond laser filtered at 620 nm (donor emission), and photons were counted for individual pixels (surface resolution approximately 300 nm) over time, allowing both total fluorescence intensity and lifetime to be analyzed (FIG. 5d). In regions with only CpcA-PEB, fluorescence intensity was above 4000 arbitrary units (a.u.) and the lifetime was over 2 ns (τₐᵥ=2058 ps). Regions with both CpcA-PEB and SP2-ZnPPa show reduced fluorescence intensity (<1000 a.u.) and lifetimes under 0.9 ns (τₐᵥ=839 ps). We estimate the energy transfer efficiency of CpcA-PEB to SP2-ZnPPa in 2D arrays to be 59%, similar to figures seen for natural fluorescent proteins (Duncan et al. 2004; Becker et al. 2004; Tramier et al. 2002).

For efficient solar energy conversion, nature organizes photosynthetic machinery into specialized compartments such as thylakoids in plants or chromatophore vesicles in purple photosynthetic bacteria (Singharoy et al. 2019). As a first step towards such structures, we sought to incorporate a Chl-binding SP protein into a two-component supercomplex with octahedral symmetry (King et al, 2014). The C₂ symmetry axes of 12 copies of the SP2 dimer and the C₃ axes of 8 copies of a C₃-symmetric homotrimer (Boyken et al. 2016) were aligned with the C₂ and C₃ axes of an octahedron. We sampled rotations and translations along these axes to generate a closely packed octahedral model with the C₂ dimers on the edges and the C₃ trimers on the vertices. Interface residues were redesigned to create binding surfaces between the SP2 dimers and the trimers. Twenty-one designs were experimentally characterized, and one was found to assemble into octahedral structures by negative-stain EM. In this nanocage, the SP2-like component shares 87% sequence identity with the original SP2 design, and its absorbance spectrum has a red-shifted shoulder in the Q_{y} region, similar to the original SP2-ZnPPaM complex (data not shown).

The cryo-EM structure of the 600 kDa octahedral nanocage with 24 ZnPPaM bound was solved to intermediate resolution (average resolution 6.5 Å) in which helices are well resolved. The design model accurately predicts protein-protein interfaces and the overall architecture (FIG. 6a,b). The asymmetric unit of the cryo-EM structure agrees with the design model to 3.4 Å backbone RMSD. Variability analysis showed several modes of flexibility which may have limited the resolution. While the resolution is not sufficient to confidently determine the orientations of the Chls, the cryo-EM density map is consistent

### Discussion

We describe the first designed proteins that hold Chl dimers in precisely defined closely juxtaposed geometries. We obtain crystal structures of two holo-state designs: the first, SP1, reproduces the binding geometry of the native purple bacterial reaction center special pair with sub-angstrom precision, and the second, SP2, has a distinct geometry with the Chls closer together. Our use of symmetry reduces the complexity of the design procedure while ensuring equivalent site energies for the two bound Chls to strengthen excitonic coupling. Symmetry also enables integration of the de novo special pair proteins into larger supercomplexes. Our octahedral nanocage incorporating 12 ZnPPaM dimers indicates de novo design of photosynthetic compartments analogous to thylakoids or chromatophores.

SP2 exhibits spectroscopic hallmarks of native special pairs including Cotton effects by CD, shifting of absorption and fluorescence bands, and energy transfer activity when paired with the native antenna protein CpcA. SP1 exhibits weaker excitonic coupling than SP2 and the BChl special pair of purple photosynthetic bacteria despite its close structural similarity to the latter. The stronger coupling of SP2 relative to SP1 may reflect the closer spacing of the ZnPPaM molecules in SP2, whereas the stronger coupling of the purple bacterial special pair is due to the stronger Q_{y} transition dipole moment of bacteriochlorins as compared to chlorins (Knox and Spring 2003). Directed evolution could alter the binding specificity for different types of chlorophylls and increase absorbance band shifts for more effective exciton trapping. Prediction of the spectroscopic properties of a Chl dimer in a protein is complicated by the fact that Chl-Chl coupling energies are typically similar in magnitude to the available thermal energy, Frank-Condon active vibrational and phonon reorganization energies, and local Chl vibrational frequencies (Reppert 2023). Accurate optical predictions require benchmarking of theoretical methods using robust model systems. Native photosynthetic proteins can be difficult to isolate and typically contain many interacting pigment molecules, creating spectral congestion. The highly thermostable, water-soluble Chl dimer proteins described herein avoid the complexity of native pigment-protein complexes and provide a testbed to investigate structure-spectrum relationships.

Studies of photosynthetic light harvesting and charge separation indicate that natural photosynthesis leaves room for efficiency improvements. The successful design of excitonically coupled chromophore pairs, and the assembly of these into organized superstructures, indicates that de novo protein design can provide new solar-to-fuel energy conversion technologies. With its red-shifted absorbance spectrum, SP2 is well tuned to accept energy from light-harvesting Chls or other pigments (FIG. 5), and it has the long-lived excited state (>3 ns fluorescence emission lifetime; data not shown) needed to allow electron transfer to occur. To couple light absorption to charge separation for solar fuel production, the next step is to engineer transfer of the excited state electron to a low potential electron acceptor.

### Methods

Computational placement of the chlorophyll special pair into symmetric protein scaffolds: Identifying residue positions capable of accommodating the Chl special pair which is scalable to millions of potential scaffolds was achieved by utilizing a motif-hash-based method (Fallas et al. 2017; Yao et al. 2022) specifically adopted for the histidine-Chl dimer motif inspired by the special pair of purple bacteria, P865. However, the number of example structures of the histidine-Chl dimer motif found in the PDB is not acceptable for effectively populating a motif hash table. Therefore, additional structural examples of the symmetric histidine-Chl dimer complex were generated de novo.

The conformer generation was achieved using the NeRF algorithm which translates internal molecular coordinates to global molecular coordinates. Various conformers were generated by varying the internal coordinates such as the relative positioning of the Chl groups, the dihedral of ligation by the histidine residue, and the rotamer of the histidine sidechain. The full complex was duplicated along the C₂ axis to create the symmetric complex. If the relative orientations of the Chls were varied, clashes between the rings and their substitutions were evaluated and filtered. The full process of de novo motif generation was repeated for ligation with the epsilon and delta nitrogen of the imidazole ring.

Once the de novo conformers were generated, the 6-D transformation that defines the relative orientation of the N-CA-C atoms of the ligating histidine residues were hashed using a method described previously (Fallas et al. 2017; Yao et al. 2022). The hashed 6-D transformation was used as a key in a multi value hash table and the associated value was a vector that defined the information necessary to rebuild the histidine-Chl complex, which nitrogen from the histidine was used for ligation and the internal coordinates of the histidine rotamer.

During evaluation of design scaffolds, the 6-D transformation of each symmetric residue pair across chains was evaluated and hashed using the same method used to hash the de novo conformers described above. That allowed the identification of symmetric residue pairs that have similar 6-D transformations to the potentially acceptable ligation geometries. If a matching 6-D transformation was found, the histidine-Chl complex was rebuilt from the associated value in the hash table, and the complex was evaluated in the context of the protein. If the Chls did not clash with the backbone atoms of the protein, the placement was accepted and passed into the protein design process.

Protein expression and purification: Synthetic genes with N-terminal His6 tags followed by TEV protease cleavage sites were purchased in pET29b expression vectors from Integrated DNA Technologies, Inc. Plasmids were transformed into Lemo21™ (DE3) Competent E. coli (New England Biolabs). For each protein, a single E. coli colony was grown in a culture of 5 mL of LB with 100 μg/mL kanamycin overnight at 37° C. Overnight cultures were used to inoculate 50-500 mL cultures of auto-induction media (Studier 2005). Bacteria were grown in auto-induction media at 37° C. with shaking for 4 hours, then incubated shaking overnight at 18° C. Bacteria were harvested and resuspended in 300 mM NaCl, 30 mM imidazole, 25 mM Tris buffer at pH 8, ˜0.01 mg/mL DNase (Sigma-Aldrich), ˜0.1 mg/mL lysozyme (Sigma-Aldrich), and Pierce™ Protease Inhibitor Tablets (Thermo Fisher Scientific). Bacteria were lysed by sonication and centrifuged at ˜18,000 g for 30 minutes. Soluble fractions were purified by Immobilized Metal Affinity Chromatography (IMAC) gravity columns packed with Ni-NTA agarose resin (Qiagen) at room temperature. Columns were washed with a buffer containing 20 mM imidazole and proteins were eluted with a 300 mM imidazole buffer. Samples were digested with His-tagged TEV protease in the presence of 0.5 mM dithiothreitol for 1-2 days at room temperature. Digested proteins were buffer exchanged into 20 mM imidazole buffer, 300 mM NaCl, and 25 mM Tris buffer at pH 8 and applied to IMAC columns to remove TEV protease and uncleaved protein. Proteins were further purified by size-exclusion chromatography (SEC) using an ÄKTA FPLC with a Superdex™ 200 Increase 10/300 GL column (GE Healthcare Life Sciences). Protein and Chl molecular weights were verified by reverse-phase liquid chromatography/mass spectrometry (LC/MS) with a G6230B TOF instrument using an AdvanceBio™ RP-Desalting column. Mass spectra were deconvoluted in Bioconfirm™ using a total entropy algorithm.

Protein-chlorophyll sample preparation: Zn pheophorbide a methyl ester (ZnPPaM) was purchased from Frontier Scientific, Inc. ZnPPaM stock solutions were prepared in dimethyl sulfoxide (DMSO) or methanol to concentrations between 200 μM and 1 mM. ZnPPaM concentrations were determined using mass measurements and by using the known absorptivity of Zn pheophytin a, which has a similar absorbance spectrum and an extinction coefficient €659 nm of 77,300 M⁻¹ cm⁻¹ in 80% acetone/20% deionized water (Jones et al. 1976). Ultraviolet/visible (UV/vis) absorbance spectra were collected using a V-750 spectrophotometer with a 1 nm bandwidth and 400 nm/min scanning speed. Protein-ZnPPaM complexes were prepared by slowly adding freshly-prepared ZnPPaM stock solution to protein solution in aqueous buffer at room temperature and incubating samples for several hours. Unbound ZnPPaM was removed by centrifugation to pellet precipitated ZnPPaM, sterile filtration using a 0.22 μm syringe filter, and/or running a PD-10 desalting column purification (Sephadex™ G-25 M resin, Cytiva Life Sciences).

Circular Dichroism (CD) spectroscopy: CD spectra were collected using a J-1500 spectrophotometer. For protein secondary structure assays, spectra were measured on samples of 0.2-0.4 mg/mL protein in 1 mm quartz cuvettes from 260 to 190 nm with a 1 nm bandwidth, 1 nm data interval, data integration time (DIT) of 1 second, and scanning speed of 50 nm/min. Thermal melts were monitored at 222 nm from 2 to 98° C. with a 2 nm bandwidth and 8 second DIT. UV/vis/near-infrared (NIR) CD transitions of protein-bound Chls were examined in the 800-300 nm region in 1 cm quartz cuvettes as averages of 10 scans using a 3 nm bandwidth, 1 nm data interval, DIT of 4 seconds, and scanning speed of 50 nm/min unless otherwise noted. UV/vis/NIR CD spectra shown in FIG. 2 were collected after sterile filtering with 0.22 μm filter and PD-10 desalting column purification (Sephadex™ G-25 M resin, Cytiva Life Sciences). Each spectrum represents the average of two independent sample preparations. Samples contained 8-15 μM protein (monomer concentration) with equimolar ZnPPaM, 150 mM NaCl, and 10 mM Tris buffer at pH 8. ZnPPaM dry powder was dissolved in methanol stock solutions immediately before adding to protein solutions. Samples were allowed to incubate for 8-16 hours at room temperature prior to measuring spectra.

Small angle X-ray scattering (SAXS): Data were collected at the Advanced Light Source (ALS) using the SIBYLS™ beamline for high throughput SAXS (Dyer et al. 2014). Proteins were sent as 30 μL samples in 96-well plates with buffer-matched blank solutions for background subtraction. Data sets were processed in SAXS Frameslice™ version 1.4.13 and compared to design models using FoXS™ (Schneidman-Duhovny et al. 2016, 2013).

X-ray crystallography for SP1 and SP2: Crystals of SP1 and SP2 were grown using protein purified as described above. Protein samples dispensed in 1 μL drops at purification concentrations were mixed with equal volume of a crystallization solution and set in hanging drops (refer to Table 6 for conditions). Vapor phase equilibration of the resulting drops against a 1 mL reservoir of the same crystallization solution resulted in growth of crystals. The crystals were flash cooled in liquid nitrogen. Diffraction data were collected on a Pilatus area detector at the Advanced Light Source (ALS) synchrotron facility at beamline 5.0.2 for SP1-ZnPPaM and SP2-ZnPPaM protein assemblies. Diffraction data were collected on a Rigaku HyPix-6000HE hybrid photon counting detector at the Fred Hutchinson Cancer Center (Fred Hutch) for SP2. The resulting data sets (Table 6) extend to 2.0 Å, 2.4 Å, and 2.5 Å resolution for SP1-ZnPPaM, apo-state SP2, and SP2-ZnPPaM, respectively. The asymmetric units of the SP1-ZnPPaM and apo-state SP2 structures each contained one complete dimer (two copies of a protein subunit), and the SP2-ZnPPaM structure had 2 dimers in the asymmetric unit.

Data were processed using HKL2000 ™ (Otwinowski and Minor 1997) or Aimless™ (Evans and Murshudov 2013). The placement of subunits was determined using the molecular replacement algorithm in program PHENIX™ (Adams et al. 2010). Local rebuilding of all constructs was performed using the program COOT (P. Emsley et al. 2010), followed by refinement in PHENIX™ (Adams et al. 2010). For the ZnPPaM-bound structures, the protein was built and refined completely with waters (excluding waters from the binding site) and other chemicals before manually fitting ZnPPaM into the density that remained. ZnPPaM energies were calculated using eLBOW (Moriarty, Grosse-Kunstleve, and Adams 2009). The final values for R_{work}/R_{free} are noted in Table 6.

X-ray crystallography for SP3x: All crystallization experiments for the SP3x protein were conducted using the sitting drop vapor diffusion method. Crystallization trials were set up in 200 nL drops using the 96-well plate format at 20° C. Crystallization plates were set up using a Mosquito™ from SPT Labtech, then imaged using UVEX™ microscopes and UVEX™ PS-600 from JAN Scientific. Diffraction quality SP3x crystals formed in 2.4 M sodium malonate dibasic monohydrate pH 7.0.

Diffraction data were collected at the Advanced Light Source at beamline 5.0.1. X-ray intensities and data reduction were evaluated and integrated using XDS (Kabsch 2010) and merged/scaled using Pointless™/Aimless™ in the CCP4 program suite (Winn et al. 2011). Structure determination and refinement starting phases were obtained by molecular replacement using Phaser™ (McCoy et al. 2007) using the designed model for the structures. Following molecular replacement, the models were improved using phenix.autobuild (Adams et al. 2010); efforts were made to reduce model bias by setting rebuild-in-place to false, and using simulated annealing and prime-and-switch phasing. Structures were refined in Phenix™ (Adams et al. 2010). Model building was performed using COOT (Paul Emsley and Cowtan 2004). The final model was evaluated using MolProbity™ (Williams et al. 2018). Data collection and refinement statistics are recorded in Table 6.

Protein structure alignment: Protein crystal structures were compared to Rosetta™ design models by aligning corresponding backbone C_{α} atoms and calculating RMSDs using TM-align (Zhang and Skolnick 2005). (B) Chl special pair geometries were compared using the align function in The PyMOL™ Molecular Graphics System, Version 2.5.2, Schrödinger, LLC. To facilitate comparison of the geometries of special pairs composed of different (B) Chl types, omit unimportant conformational differences such as rotameric states of peripheral substituents, and neglect differences in the Mg(II) vs. Zn(II) positions, only the atoms of the tetrapyrrole rings were considered in pairwise special pair structural alignments. These atoms included the 4 pyrrole nitrogen atoms, 16 pyrrole carbon atoms, and 4 methine bridge carbons from each (B) Chl monomer, giving 48 atoms per (B) Chl dimer that were used for structural comparisons. Corresponding atoms were aligned in PyMOL™ and the RMSD over all 48 atom pairs was calculated. Native BChl a special pairs used for comparison to the SP1 protein came from 5 different species of purple photosynthetic bacteria, including Rhodobacter sphaeroides, Rhodopseudomonas palustris, Thermochromatium tepidum, Gemmatimonas phototrophica, and Thiorhodovibrio strain 970. The PDB IDs of the nine X-ray crystal and cryo-EM structures containing the native special pairs used for comparison to SP1 were: 7PIL, 7VNY, 6Z27, 6Z02, 6Z5S, 3WMM, 5Y5S, 700U, and 7C9R (Cao et al. 2022; Niwa et al. 2014; Qian et al. 2021, 2022; Selikhanov et al. 2020; Swainsbury et al. 2021; Tani et al. 2020; Yu et al. 2018).

Nanocage design: The Chl binding dimer SP2 was docked against a library of trimeric cyclic oligomer scaffolds (C₃) from previous de novo designs (Fallas et al. 2017; Boyken et al. 2016; Hsia et al. 2021) to form octahedral cages (O32) using the RPXDock™ software (Sheffler et al. 2022). The RPXdock™ package utilizes a hierarchical sampling strategy to search for interfaces with high shape complementarity based on residue pair transform scoring. The top 10 scored docking configurations for each scaffold were subsequently sequence designed by symmetric Rosetta™ Design calculations, using a previously reported protocol (King et al. 2014) to carry out two-component protein-protein interface design. Briefly, we aim to design low-energy, well-packed hydrophobic protein-protein interfaces where protein building blocks are treated as rigid backbones and only side chain rotamers of interface residues are packed with layer design restrictions. The beta_nov16 or a clash-fixed score function was used during the design. Finally, all cage designs were filtered based on shape complementarity (>0.6), interface surface area (solvent-accessible surface area, 1000<sasa<1600), predicted binding energy (ddG<−20 kcal/mol), buried unsatisfied hydrogen bonds (uhb<3), and clash check (<3).

Transmission negative-stain electron microscopy (nsEM) and image processing: SEC purified cage fractions were diluted to about 0.5 μM (monomeric component concentration) for negative-stain EM characterization. Briefly, on a glow-discharged formvar/carbon supported 400-mesh copper grid (Ted Pella, Inc.), 6 μL of protein sample were drop-casted for 2 mins. The grid was blotted and stained with 3 μL of 2% uranyl formate, blotted again, and stained with 3 μL of uranyl formate for 20 s before final blotting. Micrographs of stained samples were taken on a 120 kV Talos™ L120C transmission electron microscope. All nsEM datasets were collected using the EPU software and processed by CryoSparc™ (Punjani et al. 2017) with contrast transfer function (CTF) correction. All the particle picks were 2D classified for 20 iterations into 50 classes. Particles from selected classes were used for building the ab-initio initial model. The initial model was homogeneously refined using C₁ and the corresponding O symmetry.

### REFERENCES

Adams, Paul D., Pavel V. Afonine, Gábor Bunkóczi, Vincent B. Chen, Ian W. Davis, Nathaniel Echols, Jeffrey J. Headd, et al. 2010. “PHENIX: A Comprehensive Python-Based System for Macromolecular Structure Solution.” Acta Crystallographica. Section D, Biological Crystallography 66 (Pt 2): 213-21.
Ahad, Safa, Chientzu Lin, and Mike Reppert. 2023. “Photosynthetic Protein Spectroscopy Lab.” doi.org/10.21981/0NQJ-NZ11.
Álvarez-Moreno, M., C. de Graaf, N. López, F. Maseras, J. M. Poblet, and C. Bo. 2015. “Managing the Computational Chemistry Big Data Problem: The ioChem-BD Platform.” Journal of Chemical Information and Modeling 55 (1): 95-103.
Amerongen, Herbert van, Leonas Valkunas, and Rienk van Grondelle. 2000. Photosynthetic Excitons. World Scientific.
Anandakrishnan, Ramu, Boris Aguilar, and Alexey V. Onufriev. 2012. “H++3.0: Automating pK Prediction and the Preparation of Biomolecular Structures for Atomistic Molecular Modeling and Simulations.” Nucleic Acids Research 40 (Web Server issue): W537-41.
Barber, James, and Phong D. Tran. 2013. “From Natural to Artificial Photosynthesis.” Journal of the Royal Society, Interface/the Royal Society 10 (81): 20120984.
Barnett, Samuel F. H., Andrew Hitchcock, Amit K. Mandal, Cvetelin Vasilev, Jonathan M. Yuen, James Morby, Amanda A. Brindley, et al. 2017. “Repurposing a Photosynthetic Antenna Protein as a Super-Resolution Microscopy Label.” Scientific Reports 7 (1): 16807.
Becker, W., A. Bergmann, M. A. Hink, K. König, K. Benndorf, and C. Biskup. 2004. “Fluorescence Lifetime Imaging by Time-Correlated Single-Photon Counting.” Microscopy Research and Technique 63 (1): 58-66.
Bednarczyk, Dominika, Orly Dym, Vadivel Prabahar, Yoav Peleg, Douglas H. Pike, and Dror Noy. 2016. “Fine Tuning of Chlorophyll Spectra by Protein-Induced Ring Deformation.” Angewandte Chemie 55 (24): 6901-5.
Bepler, Tristan, Andrew Morin, Micah Rapp, Julia Brasch, Lawrence Shapiro, Alex J. Noble, and Bonnie Berger. 2019. “Positive-Unlabeled Convolutional Neural Networks for Particle Picking in Cryo-Electron Micrographs.” Nature Methods 16 (11): 1153-60.
Blankenship, Robert E., David M. Tiede, James Barber, Gary W. Brudvig, Graham Fleming, Maria Ghirardi, M. R. Gunner, et al. 2011. “Comparing Photosynthetic and Photovoltaic Efficiencies and Recognizing the Potential for Improvement.” Science 332 (6031): 805-9.
Boxer, S. G., and G. L. Closs. 1976. “A Covalently Bound Dimeric Derivative of Pyrochlorophyllide A. A Possible Model for Reaction Center Chlorophyll.” Journal of the American Chemical Society 98 (17): 5406-8.
Boyken, Scott E., Zibo Chen, Benjamin Groves, Robert A. Langan, Gustav Oberdorfer, Alex Ford, Jason M. Gilmore, et al. 2016. “De Novo Design of Protein Homo-Oligomers with Modular Hydrogen-Bond Network-Mediated Specificity.” Science 352 (6286): 680-87.
Brunette, T. J., Matthew J. Bick, Jesse M. Hansen, Cameron M. Chow, Justin M. Kollman, and David Baker. 2020. “Modular Repeat Protein Sculpting Using Rigid Helical Junctions.” Proceedings of the National Academy of Sciences of the United States of America 117 (16): 8870-75.
Cao, Peng, Laura Bracun, Atsushi Yamagata, Bern M. Christianson, Tatsuki Negami, Baohua Zou, Tohru Terada, et al. 2022. “Structural Basis for the Assembly and Quinone Transport Mechanisms of the Dimeric Photosynthetic RC-LH1 Supercomplex.” Nature Communications 13 (1): 1-12.
Caram, Justin R., Sandra Doria, Dörthe M. Eisele, Francesca S. Freyria, Timothy S. Sinclair, Patrick Rebentrost, Seth Lloyd, and Moungi G. Bawendi. 2016. “Room-Temperature Micron-Scale Exciton Migration in a Stabilized Emissive Molecular Aggregate.” Nano Letters 16 (11): 6808-15.
Case, D. A., I. Y. Ben-Shalom, S. R. Brozell, D. S. Cerutti, T. E. Cheatham, III, V. W. D. Cruzeiro, et al. 2018. AMBER 2018. University of California, San Francisco.
Chen, Jing-Hua, Hangjun Wu, Caihuang Xu, Xiao-Chi Liu, Zihui Huang, Shenghai Chang, Wenda Wang, et al. 2020. “Architecture of the Photosynthetic Complex from a Green Sulfur Bacterium.” Science 370 (6519): eabb6350.
Cohen-Ofri, Ilit, Maurice van Gastel, Joanna Grzyb, Alexander Brandis, Iddo Pinkas, Wolfgang Lubitz, and Dror Noy. 2011. “Zinc-Bacteriochlorophyllide Dimers in de Novo Designed Four-Helix Bundle Proteins. A Model System for Natural Light Energy Harvesting and Dissipation.” Journal of the American Chemical Society 133 (24): 9526-35.
Croce, Roberta, and Herbert van Amerongen. 2014. “Natural Strategies for Photosynthetic Light Harvesting.” Nature Chemical Biology 10 (7): 492-501.
Curti, Mariano, Valentin Maffeis, Luís Gustavo Teixeira Alves Duarte, Saeed Shareef, Luisa Xiomara Hallado, Carles Curutchet, and Elisabet Romero. 2023. “Engineering Excitonically-Coupled Dimers in an Artificial Protein for Light Harvesting via Computational Modelling.” Protein Science: A Publication of the Protein Society, January, e4579.
Duncan, R. R., A. Bergmann, M. A. Cousin, D. K. Apps, and M. J. Shipston. 2004. “Multi-Dimensional Time-Correlated Single Photon Counting (TCSPC) Fluorescence Lifetime Imaging Microscopy (FLIM) to Detect FRET in Cells.” Journal of Microscopy 215 (Pt 1): 1-12.
Dyer, Kevin N., Michal Hammel, Robert P. Rambo, Susan E. Tsutakawa, Ivan Rodic, Scott Classen, John A. Tainer, and Greg L. Hura. 2014. “High-Throughput SAXS for the Characterization of Biomolecules in Solution: A Practical Approach.” Methods in Molecular Biology 1091:245-58.
Echols, Nathaniel, Ralf W. Grosse-Kunstleve, Pavel V. Afonine, Gábor Bunkóczi, Vincent B. Chen, Jeffrey J. Headd, Airlie J. McCoy, et al. 2012. “Graphical Tools for Macromolecular Crystallography in PHENIX.” Journal of Applied Crystallography 45 (Pt 3): 581-86.
Emsley, Paul, and Kevin Cowtan. 2004. “Coot: Model-Building Tools for Molecular Graphics.” Acta Crystallographica. Section D, Biological Crystallography 60 (Pt 12 Pt 1): 2126-32.
Emsley, P., B. Lohkamp, W. G. Scott, and K. Cowtan. 2010. “Features and Development of Coot.” Acta Crystallographica. Section D, Biological Crystallography 66 (Pt 4): 486-501.
Ennist, Nathan M., Joshua A. Mancini, Dirk B. Auman, Chris Bialas, Martin J. Iwanicki, Tatiana V. Esipova, Bohdana M. Discher, Christopher C. Moser, and P. Leslie Dutton. 2017. “Maquette Strategy for Creation of Light- and Redox-Active Proteins.” In Photosynthesis and Bioenergetics, 1-33. WORLD SCIENTIFIC.
Ennist, Nathan M., S. E. Stayrook., P. L. Dutton, and C. C. Moser. 2022. “Rational Design of Photosynthetic Reaction Center Protein Maquettes.” Frontiers in Molecular Biosciences 9 (September). doi.org/10.3389/fmolb.2022.997295.
Ennist, Nathan M., Zhenyu Zhao, Steven E. Stayrook, Bohdana M. Discher, P. Leslie Dutton, and Christopher C. Moser. 2022. “De Novo Protein Design of Photochemical Reaction Centers.” Nature Communications. doi.org/10.1038/s41467-022-32710-5.
Evans, Philip R., and Garib N. Murshudov. 2013. “How Good Are My Data and What Is the Resolution?” Acta Crystallographica. Section D, Biological Crystallography 69 (Pt 7): 1204-14.
Fallas, Jorge A., George Ueda, William Sheffler, Vanessa Nguyen, Dan E. McNamara, Banumathi Sankaran, Jose Henrique Pereira, et al. 2017. “Computational Design of Self-Assembling Cyclic Protein Homo-Oligomers.” Nature Chemistry 9 (4): 353-60.
Farid, Tammer A., Goutham Kodali, Lee A. Solomon, Bruce R. Lichtenstein, Molly M. Sheehan, Bryan A. Fry, Chris Bialas, et al. 2013. “Elementary Tetrahelical Protein Design for Diverse Oxidoreductase Functions.” Nature Chemical Biology 9 (12): 826-33.
Ferretti, Marco, Vladimir I. Novoderezhkin, Elisabet Romero, Ramunas Augulis, Anjali Pandit, Donatas Zigmantas, and Rienk van Grondelle. 2014. “The Nature of Coherences in the B820 Bacteriochlorophyll Dimer Revealed by Two-Dimensional Electronic Spectroscopy.” Physical Chemistry Chemical Physics: PCCP 16 (21): 9930-39.
Frisch, M. J., G. W. Trucks, H. B. Schlegel, G. E. Scuseria, M. A. Robb, J. R. Cheeseman, G. Scalmani, et al. 2016. Gaussian 16 (version Revision B.01). Wallingford, CT.
Fry, H. Christopher, Andreas Lehmann, Jeffery G. Saven, William F. DeGrado, and Michael J. Therien. 2010. “Computational Design and Elaboration of a de Novo Heterotetrameric Alpha-Helical Protein That Selectively Binds an Emissive Abiological (porphinato) zinc Chromophore.” Journal of the American Chemical Society 132 (11): 3997-4005.
Gisriel, Christopher, Iosifina Sarrou, Bryan Ferlez, John H. Golbeck, Kevin E. Redding, and Raimund Fromme. 2017. “Structure of a Symmetric Photosynthetic Reaction Center-Photosystem.” Science 357 (6355): 1021-25.
Gordon, John C., Jonathan B. Myers, Timothy Folta, Valia Shoja, Lenwood S. Heath, and Alexey Onufriev. 2005. “H++: A Server for Estimating pKas and Adding Missing Hydrogens to Macromolecules.” Nucleic Acids Research 33 (Web Server issue): W368-71.
Gorka, Michael, Amgalanbaatar Baldansuren, Amanda Malnati, Elijah Gruszecki, John H. Golbeck, and K. V. Lakshmi. 2021. “Shedding Light on Primary Donors in Photosynthetic Reaction Centers.” Frontiers in Microbiology 12 (October): 735666.
Hicks, Derrick R., Madison A. Kennedy, Kirsten A. Thompson, Michelle DeWitt, Brian Coventry, Alex Kang, Asim K. Bera, et al. 2022. “De Novo Design of Protein Homodimers Containing Tunable Symmetric Protein Pockets.” Proceedings of the National Academy of Sciences 119 (30): e2113400119.
Hitchcock, Andrew, Christopher Neil Hunter, Roman Sobotka, Josef Komenda, Marcel Dann, and Dario Leister. 2022. “Redesigning the Photosynthetic Light Reactions to Enhance Photosynthesis—the PhotoRedesign Consortium.” The Plant Journal: For Cell and Molecular Biology 109 (1): 23-34.
Hsia, Yang, Rubul Mout, William Sheffler, Natasha I. Edman, Ivan Vulovic, Young-Jun Park, Rachel L. Redler, et al. 2021. “Design of Multi-Scale Protein Complexes by Hierarchical Building Block Fusion.” Nature Communications 12 (1): 2294.
Huang, Xia, Cvetelin Vasilev, and C. Neil Hunter. 2020. “Excitation Energy Transfer between Monomolecular Layers of Light Harvesting LH2 and LH1-Reaction Centre Complexes Printed on a Glass Substrate.” Lab on a Chip 20 (14): 2529-38.
Iozzi, Maria Francesca, Benedetta Mennucci, Jacopo Tomasi, and Roberto Cammi. 2004. “Excitation Energy Transfer (EET) between Molecules in Condensed Matter: A Novel Application of the Polarizable Continuum Model (PCM).” The Journal of Chemical Physics 120 (15): 7029-40.
Jones, I. D., R. C. White, E. Gibbs, and L. S. Butler. 1976. “Estimation of Zinc Pheophytins, Chlorophylls, and Pheophytins in Mixtures in Diethyl Ether or 80% Acetone by Spectrophotometry and Fluorometry.” Journal of Agricultural and Food Chemistry 25 (1): 146-49.
Jorgensen, William L., Jayaraman Chandrasekhar, Jeffry D. Madura, Roger W. Impey, and Michael L. Klein. 1983. “Comparison of Simple Potential Functions for Simulating Liquid Water.” The Journal of Chemical Physics 79 (2): 926-35.
Jurinovich, Sandro, Lorenzo Cupellini, Ciro A. Guido, and Benedetta Mennucci. 2018. “EXAT: EXcitonic Analysis Tool.” Journal of Computational Chemistry 39 (5): 279-86.
Jurinovich, Sandro, Ciro A. Guido, Torsten Bruhn, Gennaro Pescitelli, and Benedetta Mennucci. 2015. “The Role of Magnetic-electric Coupling in Exciton-Coupled ECD Spectra: The Case of Bis-Phenanthrenes.” Chemical Communications 51 (52): 10498-501.
Jurinovich, Sandro, Gennaro Pescitelli, Lorenzo Di Bari, and Benedetta Mennucci. 2014. “A TDDFT/MMPol/PCM Model for the Simulation of Exciton-Coupled Circular Dichroism Spectra.” Physical Chemistry Chemical Physics: PCCP 16 (31): 16407-18.
Kabsch, Wolfgang. 2010. “XDS.” Acta Crystallographica. Section D, Biological Crystallography 66 (Pt 2): 125-32.
Kim, Henry S., Anne Martel, Eric Girard, Martine Moulin, Michael Härtlein, Dominique Madern, Martin Blackledge, Bruno Franzetti, and Frank Gabel. 2016. “SAXS/SANS on Supercharged Proteins Reveals Residue-Specific Modifications of the Hydration Shell.” Biophysical Journal 110 (10): 2185-94.
King, Neil P., Jacob B. Bale, William Sheffler, Dan E. McNamara, Shane Gonen, Tamir Gonen, Todd O. Yeates, and David Baker. 2014. “Accurate Design of Co-Assembling Multi-Component Protein Nanomaterials.” Nature 510 (7503): 103-8.
Knox, Robert S., and Bryan Q. Spring. 2003. “Dipole Strengths in the Chlorophylls.” Photochemistry and Photobiology 77 (5): 497-501.
Kobuke, Yoshiaki, and Hidekazu Miyaji. 1994. “Supramolecular Organization of Imidazolyl-Porphyrin to a Slipped Cofacial Dimer.” Journal of the American Chemical Society 116 (9): 4111-12.
Kodali, Goutham, Joshua A. Mancini, Lee A. Solomon, Tatiana V. Episova, Nicholas Roach, Christopher J. Hobbs, Pawel Wagner, et al. 2017. “Design and Engineering of Water-Soluble Light-Harvesting Protein Maquettes.” Chemical Science 8 (1): 316-24.
Lander, Gabriel C., Scott M. Stagg, Neil R. Voss, Anchi Cheng, Denis Fellmann, James Pulokas, Craig Yoshioka, et al. 2009. “Appion: An Integrated, Database-Driven Pipeline to Facilitate EM Image Processing.” Journal of Structural Biology 166 (1): 95-102.
Lindorfer, Dominik, Frank Müh, and Thomas Renger. 2017. “Origin of Non-Conservative Circular Dichroism of the CP29 Antenna Complex of Photosystem II.” Physical Chemistry Chemical Physics: PCCP 19 (11): 7524-36.
Lin, X., H. A. Murchison, V. Nagarajan, W. W. Parson, J. P. Allen, and J. C. Williams. 1994. “Specific Alteration of the Oxidation Potential of the Electron Donor in Reaction Centers from Rhodobacter sphaeroides.” Proceedings of the National Academy of Sciences of the United States of America 91 (22): 10265-69.
Li, Pengfei, and Kenneth M. Merz Jr. 2016. “MCPB.py: A Python Based Metal Center Parameter Builder.” Journal of Chemical Information and Modeling 56 (4): 599-604.
Madjet, M. E., A. Abdurahman, and T. Renger. 2006. “Intermolecular Coulomb Couplings from Ab Initio Electrostatic Potentials: Application to Optical Transitions of Strongly Coupled Pigments in Photosynthetic Antennae and Reaction Centers.” The Journal of Physical Chemistry. B 110 (34): 17268-81.
Maguire, Jack B., Hugh K. Haddox, Devin Strickland, Samer F. Halabiya, Brian Coventry, Jermel R. Griffin, Surya V. S. R. K. Pulavarti, et al. 2021. “Perturbing the Energy Landscape for Improved Packing during Computational Protein Design.” Proteins 89 (4): 436-49.
Maier, James A., Carmenza Martinez, Koushik Kasavajhala, Lauren Wickstrom, Kevin E. Hauser, and Carlos Simmerling. 2015. “ff14SB: Improving the Accuracy of Protein Side Chain and Backbone Parameters from ff99SB.” Journal of Chemical Theory and Computation 11 (8): 3696-3713.
McCleese, Christopher, Zhanqian Yu, Nopondo N. Esemoto, Charles Kolodziej, Buddhadev Maiti, Srijana Bhandari, Barry D. Dunietz, Clemens Burda, and Marcin Ptaszek. 2018. “Excitonic Interactions in Bacteriochlorin Homo-Dyads Enable Charge Transfer: A New Approach to the Artificial Photosynthetic Special Pair.” The Journal of Physical Chemistry. B 122 (14): 4131-40.
McCoy, Airlie J., Ralf W. Grosse-Kunstleve, Paul D. Adams, Martyn D. Winn, Laurent C. Storoni, and Randy J. Read. 2007. “Phaser Crystallographic Software.” Journal of Applied Crystallography 40 (Pt 4): 658-74.
Mirkovic, Tihana, Evgeny E. Ostroumov, Jessica M. Anna, Rienk van Grondelle, Govindjee, and Gregory D. Scholes. 2017. “Light Absorption and Energy Transfer in the Antenna Complexes of Photosynthetic Organisms.” Chemical Reviews 117 (2): 249-93.
Moriarty, Nigel W., Ralf W. Grosse-Kunstleve, and Paul D. Adams. 2009. “Electronic Ligand Builder and Optimization Workbench (eLBOW): A Tool for Ligand Coordinate and Restraint Generation.” Acta Crystallographica. Section D, Biological Crystallography 65 (Pt 10): 1074-80.
Moser, C. C., M. M. Sheehan, N. M. Ennist, G. Kodali, C. Bialas, M. T. Englander, B. M. Discher, and P. L. Dutton. 2016. “De Novo Construction of Redox Active Proteins.” Methods in Enzymology. doi.org/10.1016/bs.mie.2016.05.048.
Myers, Jonathan, Greg Grothaus, Shivaram Narayanan, and Alexey Onufriev. 2006. “A Simple Clustering Algorithm Can Be Accurate Enough for Use in Calculations of pKs in Macromolecules.” Proteins 63 (4): 928-38.
Niwa, Satomi, Long-Jiang Yu, Kazuki Takeda, Yu Hirano, Tomoaki Kawakami, Zheng-Yu Wang-Otomo, and Kunio Miki. 2014. “Structure of the LH1-RC Complex from Thermochromatium tepidum at 3.0 Å.” Nature 508 (7495): 228-32.
Otwinowski, Z., and W. Minor. 1997. “Processing of X-Ray Diffraction Data Collected in Oscillation Mode.” Methods in Enzymology 276:307-26.
Pettersen, Eric F., Thomas D. Goddard, Conrad C. Huang, Gregory S. Couch, Daniel M. Greenblatt, Elaine C. Meng, and Thomas E. Ferrin. 2004. “UCSF Chimera—a Visualization System for Exploratory Research and Analysis.” Journal of Computational Chemistry 25 (13): 1605-12.
Pieper, J., M. Rätsep, I. Trostmann, F-J Schmitt, C. Theiss, H. Paulsen, H. J. Eichler, A. Freiberg, and G. Renger. 2011. “Excitonic Energy Level Structure and Pigment-Protein Interactions in the Recombinant Water-Soluble Chlorophyll Protein. II. Spectral Hole-Burning Experiments.” The Journal of Physical Chemistry. B 115 (14): 4053-65.
Pirro, Fabio, Nathan Schmidt, James Lincoff, Zachary X. Widel, Nicholas F. Polizzi, Lijun Liu, Michael J. Therien, et al. 2020. “Allosteric Cooperation in a de Novo-Designed Two-Domain Protein.” Proceedings of the National Academy of Sciences 117 (52): 33246-53.
Polizzi, Nicholas F., Yibing Wu, Thomas Lemmin, Alison M. Maxwell, Shao-Qing Zhang, Jeff Rawson, David N. Beratan, Michael J. Therien, and William F. DeGrado. 2017. “De Novo Design of a Hyperstable Non-Natural Protein-ligand Complex with Sub-Å Accuracy.” Nature Chemistry. doi.org/10.1038/nchem.2846.
Punjani, Ali, and David J. Fleet. 2021. “3D Variability Analysis: Resolving Continuous Flexibility and Discrete Heterogeneity from Single Particle Cryo-EM.” Journal of Structural Biology 213 (2): 107702.
Punjani, Ali, John L. Rubinstein, David J. Fleet, and Marcus A. Brubaker. 2017. “cryoSPARC: Algorithms for Rapid Unsupervised Cryo-EM Structure Determination.” Nature Methods 14 (3): 290-96.
Qian, Pu, Alastair T. Gardiner, Ivana Šímová, Katerina Naydenova, Tristan I. Croll, Philip J. Jackson, Null Nupur, et al. 2022. “2.4-Å Structure of the Double-Ring Gemmatimonas phototrophica Photosystem.” Science Advances 8 (7): eabk3139.
Qian, Pu, C. Alistair Siebert, Peiyi Wang, Daniel P. Canniffe, and C. Neil Hunter. 2018. “Cryo-EM Structure of the Blastochloris viridis LH1-RC Complex at 2.9 Å.” Nature 556 (7700): 203-8.
Qian, Pu, David J. K. Swainsbury, Tristan I. Croll, Jack H. Salisbury, Elizabeth C. Martin, Philip J. Jackson, Andrew Hitchcock, Pablo Castro-Hartmann, Kasim Sader, and C. Neil Hunter. 2021. “Cryo-EM Structure of the Monomeric Rhodobacter sphaeroides RC-LH1 Core Complex at 2.5 Å.” Biochemical Journal 478 (20): 3775-90.
Rabanal, Francesc, William F. DeGrado, and P. Leslie Dutton. 1996. “Toward the Synthesis of a Photosynthetic Reaction Center Maquette: A Cofacial Porphyrin Pair Assembled between Two Subunits of a Synthetic Four-Helix Bundle Multiheme Protein.” Journal of the American Chemical Society 118 (2): 473-74.
Rätsep, Margus, Jörg Pieper, Klaus-Dieter Irrgang, and Arvi Freiberg. 2008. “Excitation Wavelength-Dependent Electron-Phonon and Electron-Vibrational Coupling in the CP29 Antenna Complex of Green Plants.” The Journal of Physical Chemistry. B 112 (1): 110-18.
Renger, Thomas, and Rudolph A. Marcus. 2002. “On the Relation of Protein Dynamics and Exciton Relaxation in Pigment—Protein Complexes: An Estimation of the Spectral Density and a Theory for the Calculation of Optical Spectra.” The Journal of Chemical Physics 116 (22): 9997-10019.
Reppert, Mike. 2023. “Bioexcitons by Design: How Do We Get There?” The Journal of Physical Chemistry. B, February. doi.org/10.1021/acs.jpcb.2c08787.
Roe, Daniel R., and Bernard R. Brooks. 2020. “A Protocol for Preparing Explicitly Solvated Systems for Stable Molecular Dynamics Simulations.” The Journal of Chemical Physics 153 (5): 054123.
Romero, Elisabet, Vladimir I. Novoderezhkin, and Rienk van Grondelle. 2017. “Quantum Design of Photosynthesis for Bio-Inspired Solar-Energy Conversion.” Nature 543 (7645): 355-65.
Schneidman-Duhovny, Dina, Michal Hammel, John A. Tainer, and Andrej Sali. 2013. “Accurate SAXS Profile Computation and Its Assessment by Contrast Variation Experiments.” Biophysical Journal 105 (4): 962-74.
2016. “FoXS, FoXSDock and MultiFoXS: Single-State and Multi-State Structural Modeling of Proteins and Their Complexes Based on SAXS Profiles.” Nucleic Acids Research 44 (W1): W424-29.
Selikhanov, Georgii, Tatiana Fufina, Lyudmila Vasilieva, Christian Betzel, and Azat Gabdulkhakov. 2020. “Novel Approaches for the Lipid Sponge Phase Crystallization of the Rhodobacter sphaeroides Photosynthetic Reaction Center.” IUCrJ 7 (Pt 6): 1084-91.
Sener, Melih K., Deyu Lu, Thorsten Ritz, Sanghyun Park, Petra Fromme, and Klaus Schulten. 2002. “Robustness and Optimality of Light Harvesting in Cyanobacterial Photosystem I.” The Journal of Physical Chemistry. B 106 (32): 7948-60.
Şener, Melih, Johan Strümpfer, Jen Hsin, Danielle Chandler, Simon Scheuring, C. Neil Hunter, and Klaus Schulten. 2011. “Förster Energy Transfer Theory as Reflected in the Structures of Photosynthetic Light-Harvesting Systems.” Chemphyschem: A European Journal of Chemical Physics and Physical Chemistry 12 (3): 518-31.
Sharma, Vinay K., Atif Mahammed, Amir Mizrahi, Maryann Morales, Natalia Fridman, Harry B. Gray, and Zeev Gross. 2021. “Dimeric Corrole Analogs of Chlorophyll Special Pairs.” Journal of the American Chemical Society 143 (25): 9450-60.
Sheffler, William, Erin C. Yang, Quinton Dowling, Yang Hsia, Chelsea N. Fries, Jenna Stanislaw, Mark Langowski, et al. 2022. “Fast and Versatile Sequence-Independent Protein Docking for Nanomaterials Design Using RPXDock.” bioRxiv. doi.org/10.1101/2022.10.25.513641.
Singharoy, Abhishek, Christopher Maffeo, Karelia H. Delgado-Magnero, David J. K. Swainsbury, Melih Sener, Ulrich Kleinekathöfer, John W. Vant, et al. 2019. “Atoms to Phenotypes: Molecular Design Principles of Cellular Energy Metabolism.” Cell 179 (5): 1098-1111.e23.
Srivastava, Amit, Safa Ahad, Jacob H. Wat, and Mike Reppert. 2021. “Accurate Prediction of Mutation-Induced Frequency Shifts in Chlorophyll Proteins with a Simple Electrostatic Model.” The Journal of Chemical Physics 155 (15): 151102.
Studier, F. William. 2005. “Protein Production by Auto-Induction in High Density Shaking Cultures.” Protein Expression and Purification 41 (1): 207-34.
Suloway, Christian, James Pulokas, Denis Fellmann, Anchi Cheng, Francisco Guerra, Joel Quispe, Scott Stagg, Clinton S. Potter, and Bridget Carragher. 2005. “Automated Molecular Microscopy: The New Leginon System.” Journal of Structural Biology 151 (1): 41-60.
Svergun, D. I., S. Richard, M. H. Koch, Z. Sayers, S. Kuprin, and G. Zaccai. 1998. “Protein Hydration in Solution: Experimental Observation by X-Ray and Neutron Scattering.” Proceedings of the National Academy of Sciences of the United States of America 95 (5): 2267-72.
Swainsbury, David J. K., Pu Qian, Andrew Hitchcock, and C. Neil Hunter. 2023. “The Structure and Assembly of Reaction Centre-Light-Harvesting 1 Complexes in Photosynthetic Bacteria.” Bioscience Reports 43 (5). doi.org/10.1042/BSR20220089.
Swainsbury, David J. K., Pu Qian, Philip J. Jackson, Kaitlyn M. Faries, Dariusz M. Niedzwiedzki, Elizabeth C. Martin, David A. Farmer, et al. 2021. “Structures of Rhodopseudomonas palustris RC-LH1 Complexes with Open or Closed Quinone Channels.” Science Advances 7 (3): eabe2631.
Tani, Kazutoshi, Ryo Kanno, Yuki Makino, Malgorzata Hall, Mizuki Takenouchi, Michie Imanishi, Long-Jiang Yu, et al. 2020. “Cryo-EM Structure of a Ca2+-Bound Photosynthetic LH1-RC Complex Containing Multiple αβ-Polypeptides.” Nature Communications 11 (1): 4955.
Taylor, Natasha, and Ivan Kassal. 2019. “Why Are Photosynthetic Reaction Centres Dimeric?” Chemical Science 10 (41): 9576-85.
Tomasi, Jacopo, Benedetta Mennucci, and Roberto Cammi. 2005. “Quantum Mechanical Continuum Solvation Models.” Chemical Reviews 105 (8): 2999-3093.
Tramier, Marc, Isabelle Gautier, Tristan Piolot, Sylvie Ravalet, Klaus Kemnitz, Jacques Coppey, Christiane Durieux, Vincent Mignotte, and Maïté Coppey-Moisan. 2002. “Picosecond-Hetero-FRET Microscopy to Probe Protein-Protein Interactions in Live Cells.” Biophysical Journal 83 (6): 3570-77.
Wahadoszamen, Md, Iris Margalit, Anjue Mane Ara, Rienk van Grondelle, and Dror Noy. 2014. “The Role of Charge-Transfer States in Energy Transfer and Dissipation within Natural and Artificial Bacteriochlorophyll Proteins.” Nature Communications 5 (October): 5287.
Wasielewski, M. R., M. H. Studier, and J. J. Katz. 1976. “Covalently Linked Chlorophyll a Dimer: A Biomimetic Model of Special Pair Chlorophyll.” Proceedings of the National Academy of Sciences of the United States of America 73 (12): 4282-86.
Weber, G., and F. W. J. Teale. 1957. “Determination of the Absolute Quantum Yield of Fluorescent Solutions.” Transactions of the Faraday Society 53 (0): 646-55.
Williams, Christopher J., Jeffrey J. Headd, Nigel W. Moriarty, Michael G. Prisant, Lizbeth L. Videau, Lindsay N. Deis, Vishal Verma, et al. 2018. “MolProbity: More and Better Reference Data for Improved All-Atom Structure Validation.” Protein Science: A Publication of the Protein Society 27 (1): 293-315.
Winn, Martyn D., Charles C. Ballard, Kevin D. Cowtan, Eleanor J. Dodson, Paul Emsley, Phil R. Evans, Ronan M. Keegan, et al. 2011. “Overview of the CCP4 Suite and Current Developments.” Acta Crystallographica. Section D, Biological Crystallography 67 (Pt 4): 235-42.
Wright, Colin A., and Roderick K. Clayton. 1974. “The Absolute Quantum Efficiency of Bacteriochlorophyll Photooxidation in Reaction Centres of Rhodopseudomonas spheroides.” Biochimica et Biophysica Acta (BBA)—Bioenergetics. doi.org/10.1016/0005-2728 (74) 90009-7.
Yao, Sicong, Adam Moyer, Yiwu Zheng, Yang Shen, Xiaoting Meng, Chong Yuan, Yibing Zhao, Hongwei Yao, David Baker, and Chuanliu Wu. 2022. “De Novo Design and Directed Folding of Disulfide-Bridged Peptide Heterodimers.” Nature Communications 13 (1): 1-10.
Yu, Long-Jiang, Michihiro Suga, Zheng-Yu Wang-Otomo, and Jian-Ren Shen. 2018. “Structure of Photosynthetic LH1-RC Supercomplex at 1.9 Å Resolution.” Nature 556 (7700): 209-13.
Zhang, Yang, and Jeffrey Skolnick. 2005. “TM-Align: A Protein Structure Alignment Algorithm Based on the TM-Score.” Nucleic Acids Research 33 (7): 2302-9.
Zheng, Shawn Q., Eugene Palovcak, Jean-Paul Armache, Kliment A. Verba, Yifan Cheng, and David A. Agard. 2017. “MotionCor2: Anisotropic Correction of Beam-Induced Motion for Improved Cryo-Electron Microscopy.” Nature Methods 14 (4): 331-32.

TABLE 3
Amino acid sequences of designed chlorophyll special pair
proteins. Shown are the protein sequences after expression using a pET-
29b (+) vector and cleavage of N-terminal MGHHHHHHGSGSGENLYFQ sequence by
TEV protease.

| SP | |
|---|---|
| design | Amino acid sequence (after TEV protease digestion) |
| SP1 | GGDSRELIARYQILLAELAAIRADIAAERTGDPYVRKLARELKRLAQEAAEEVKRDPSSSDVN |
| | MALLLILLMIELAVRALEAAERTGDPEVRELAAELVWLAVEAAEEVQRNPSSSDVWLALHLIM |
| | LAIWAAVAALEAAERTGDPEVRELARELVRLAVEAAEEVQRNPSSKEVYMALLLILIAILEAV |
| | LSLLRAERSGDPEKREKARERVREAVERAEEVQR (SEQ ID NO: 20) |
| SP2 | SGSGSSDEEFKFLATEAKMLITAAERLAGTDPELQEMVALIKKELEQAERTFRNGDKSEAQRQ |
| | LEFVLTAARAVMNVAAAANAAGTDPELIEMVLRILKQLKEAIRTFQNGDQEEAETQLRFVLRA |
| | AIAVAVVAAALVLAGTDPELQEMVKQILEELKQAIETFARGDKEKALTQLLEVAWAAHAVAMI |
| | AAAANLAGTDPRLQQQVKEILEKLKEAIETFQKGDEEQAFRQLAEVLAEAALVALRAALTN |
| | (SEQ ID NO: 21) |
| SP3 | SGSSEELARESAEAAWRLAQAGTRLMLAWIRGDLKEIAEALIELARAVQELARVAKEYGNDEL |
| | AKTAALLAAHVAMLAILVLIRAIKEGDDEVRELAKTAIKLASTAAKIVLDALPTAEEVRQITL |
| | LAKLAEEAADKKNEDSALAVGIAAAAVLLALLALEAAQKAGIEEAEKGARLLLKLAMDAARKK |
| | NPEEALAVFNAALDVSIALKLLQSAKRAGSEETRKLAEEMLRQALERARK (SEQ ID NO: |
| | 22) |
| SP3x | SGSSEELARESAEAAWRLAQASTRATLAMIRGDLKELAEALIELARAVQELARVAKEYGNDEL |
| | AKTAALLAAHVAMLAIWVLIRAIKEGDDEVRELAKTAIKLASTAAKIVLDALPTAEEVRQITL |
| | LAKLAEEAADKKNEDSALAVGIAAIAVIIALWALEAAQKAGIEEAEKGARLLLKLAMDAARKK |
| | NPEEALAVLNAALDVSIALQLLQSAKRAGSEETRKLAEEMLRQALERARKKN (SEQ ID |
| | NO: 23) |
| JA | SGSGSWEEEAADKAIEIAEKVFRLKKSGTSEDEIAEEVAREISEVIRTLKESGSSEEVIALAL |
| | LMIILGVVMALIANGVSIREQLDISATILYEVAREQGNEEALEAIEEITRLAEEIKEEGGSIL |
| | AAHILTMAGLLVAALPSEEAAEVAKEIAKAVKAAVEAEKDGKSDEAMLALIRLILAIMAALEN |
| | RSKYSLEMVRRIAKEAEQLAEDAREGK (SEQ ID NO: 24) |
| JB | SGSTEELKKVLEDVRELAERAKESTDPEEALIIAILVIIRALAAVALDPSEEASTAVKIAIKI |
| | AEEVAKRVSDSELSASIALLIANLALVAAKAAGDEELSKLALRLAKLAAELAKDALRIAEERG |
| | NAWQAAIAVGVVVMVATILLALAKQLGSEEALKEALEVAEEAARLAKRQLELAERVGDPIVAL |
| | AAVGHVEMVAMLLAQIARESGSEEAKERAKRVAEEAEELAERVYELMRREGK (SEQ ID |
| | NO: 25) |
| JC | SGSTEKLKKVLERVRELAERAKESTSPEEALIIALLVSSLAIAAVMLDPSEEASTAVKIAIKI |
| | AEEVAKRVSDSQLSASIALSIASLALVAAEAAGDEELSKLALRLAKLAAELAKDALRIAEERG |
| | NAIDAAIAVGIVVMVAAILLQLAKLLGSEEALKEALEVAEEAARLAKRVLELAERVGDPQVAL |
| | AAVGHVQMVAILLAQIARESGSEEAKERAERVARESMELAERVLELMRREGKW (SEQ ID |
| | NO: 26) |
| JD | SGSSDEEYARMSAELAEEAAKEALEQAKREGDEDARRVAEELEKQAEEARRKKDFDLANLVLM |
| | AAIILLLALIILELAKKAGNEQMEASGLSLLATAALLAAAGGENSEDARKALEAALRLAERAT |
| | EVLEKIAKKGETALEKAIAAGTLVMLIQIIIQIAELLSKLGDTEEAKKVLDIAIELIKRVTEI |
| | LERIAKKADTDILAQAAAGHLIYLIQLLMQISILLRRLGNIDEAFKVLEEARELAERVRELLE |
| | RIAKNSDK (SEQ ID NO: 27) |
| JE | SGSGSSDELAALFLIISAKSQAWQAQSAAERTGDPRVRELARELKRLAQEAAEEVKRDPSSSE |
| | VNKNLQLISMAIALAVLALQLARKAGDEEARDIAREAVEVAVRAAQLILEGRDEQRAQTLFLI |
| | AHALLMAVMAARLGNSEVAELAVKVLRLAKELEKLLPSEIALFVVLMIALAALSAAMAASGGQ |
| | EDYARDALRRLEEAIREAEENRSKESLEKVIEEAIEAMLQANRAQTGG (SEQ ID NO: |
| | 28) |
| JF | SGSGSSDELQARFLILSAKLQAEMAQIAAERTGDPRVRELARELKRLAQEAAEEVKRDPSSSE |
| | VNSNLALISMAIALAVLALLLARKAGDEEARDIAREAVEVAVRAAELILEGRDDQRAWTLWLI |
| | AHALLMAVYAARLGNSEVAELAVKVLRLAKELEKLLPSEEALFVVSMIGLAALQAAMAAAGGQ |
| | EDYARDALRRLEEAIREAEENRSKESLEKVEEEGIEAMEQALRAIAGG (SEQ ID NO: |
| | 29) |
| JG | SGSGSSDELEARLLIISANLQAEMAQIAAERTGDPRVRELARELKRLAQEAAEEVKRDPSSSE |
| | VNQNLTLISMAIALAVLALLLARKAGDEEARDIAREAVEVAVRAAELILEGRDEQRAWTLFLI |
| | AHALLLAVLAARLGNSEVAELAVKVLRLAKELEKLLPSEIALFVVSMIGMAALQAAMAASGGQ |
| | EDYARDALRRLEEAIREAEENRSKESLEKVMEEAIRAYEQALRALAGG (SEQ ID NO: |
| | 30) |
| JH | SGSSEELAREAREAAIRLAQASTKLMEAMIRGDLKELAEALIELARAVQELARVAKEYGNDEL |
| | AKLAAFLAAHIAMLAIWVLILAIREGDDEVRELAKTAIKLASTAAKIVLDALPTAEEVRIITL |
| | LAKLAEEAADKKNEDSALAVLIAAMAVLIALAALEAAQRAGIEEAEKAARLLLKLAGEAARKK |
| | NPEEAKAVYNAALLVSIALLLLQKAKRAGSEETRRLAEEMLRQALERARKKN (SEQ ID |
| | NO: 31) |
| D2K | SGSGSREKELKQLISNLQILLLILLSQDPNNELVRKLQEKIKEAEEQGDPRRLKLIWWILALI |
| | NILQALEQEDPNNELVKRLKELIKRALEEGDPRLLQLILLILHLIWLLQALEKEDPNNELVKR |
| | LKKLIEEALKEGDPRKLWQILALLLWIAQIQKLKKQDPNNELIKRAEETLEQSLKT (SEQ |
| | ID NO: 32) |
| D2L | SGSGSREKELKQLISNLQILLLILLSEDPNNELVRKLQEKIKEAEEQGDPRRLKLIWWILNLI |
| | QMLQQLEQEDPNNELVKRLKELIKRALEEGDPRLLLLIHIILALIWLLQALEKEDPNNELVKR |
| | LKKLIEEALKEGDPRKLIQIALLLTWIATIQRLKKQDPNNELIKRAEETLEQSLKT (SEQ |
| | ID NO: 33) |
| D2M | SGSGSEEEQIRLAQLAIQIIQYQVEKNGDEQLLRLIQEITEQLQKEGDERLIAILSTMFGIIW |
| | ILEQVEKNGDEELLRLIQEITEKLARQGDERLIQIGAAMLAIIIILEQVKKNGDEQLLRLIRE |
| | ITRKLAEEGDQRLIWILHAMMAIIMSSEELEKNGDEELLQKLRELLEKIAKE (SEQ ID |
| | NO: 34) |
| D2N | SGSGSEEEEIRLAQLTIQIIQYQVEKNGDEQLLRLIQEITEQLQKEGDERLIRILNAMFTIIW |
| | ILEAVEKNGDEELLRLIQEITEKLARQGDERLIKILSAMAGIIWILEIVKKNGDEQLLRLIRE |
| | ITRKLAEEGDERLIQILANMHSIIISSEELEKNGDEELLQKLRELLEKIAKELEHHHHHH |
| | (SEQ ID NO: 35) |
| D2O | SGSGSEEEEIRLAQLAIQIIQYQVEKNGDEQLLRLIQEITEQLQKEGDERLIRILLAMFAIIL |
| | ILEHVEKNGDEELLRLIQEITEKLARQGDERLIRILIAMAGIIWILELVKKNGDEQLLRLIRE |
| | ITRKLAEEGDERLIQILLAMHAIIISSEQLEKNGDEELLQKLRELLEKIAKE (SEQ ID |
| | NO: 36) |
| D2P | SGSGSEEEEKRLIEKMSKFIWWFAQQLGDERILRAVEKLQQEIKENSPTPETFMTLINLLLSL |
| | LIWWAAQKLGDEKILEAVERLLQQIEENSPTPETHMTLAALAQSLAIWQQAQKLGDEDILEAV |
| | ERLLEEIQRNSPTPSTLIKLTWLLLLLNNWQELQKQGDEEQLRSSEKAMKELKE (SEQ ID |
| | NO: 37) |
| D2Q | SGSGSEEEEKRLIEKMSKFIWWFAQQLGDERILRAVEKLQQEIKENSPTPETYMTLGNLLLSL |
| | LIWWAAQQLGDEKILEAVERLLQQIEENSPTPETHLTLAILMSALAIWQQAQKLGDEDILEAV |
| | ERLLEEIQRNSPTPSTMTKLLWLALLLNNWEELQKQGDEEQLRSSEKAMKELKE (SEQ ID |
| | NO: 38) |
| D2R | SGSGSEEEELKRLQTLIKYLQLLIQNAKWQNPDDELLRQVEELIEEMKKRLEENPNSQEDQAL |
| | LKLLITIIALIIMILWLKSENPDDELLRQVEELIKEMIERLKENPNSQEDQALLQLLHAILGL |
| | ILLIQQLKKQNPDDELLQQVEELIEEMIKRLKENPNSEKDQILLSFLALSLLSIFLIQTQKKQ |
| | NPDDERLKQWEKQIKESIESSKE (SEQ ID NO: 39) |
| D2S | SGSGSEEEELERLKEQLKIWQKLIKLILEQDPNDEQIRRILEQAEEIIQKLQEDPNNEELQRL |
| | IQLLFNLVAIIFSIKQILELDPNDEQIRRILEQAKEIIQKLQEDPNNEELFLLAHMLFALVAI |
| | ILLIKLMLEQDPNDELIREILEQAKEIIQKLQEDPNNAELAMLIALLLSLVLWIWFMKFQLEQ |
| | DPNDEEIRRQLEQLEERIRRTLE (SEQ ID NO: 40) |
| D2T | SGSGSEEEELERLKEQLKIWQKLIKLILEQDPNDEQIRRILEQAEEIIQKLQEDPNNEELQRL |
| | IQLLFQLVLGIFIIKQILELDPNDEQIRRILEQAKEIIQKLQEDPNNEELYRLQGLLGMLVGI |
| | ILLIKLMLEQDPNDELIREILEQAKEIIQKLQEDPNNQELLSLIHLLLALVLWIWFMKFQLEQ |
| | DPNDEEIRRQLEQLEERIRRTLE (SEQ ID NO: 41) |
| tj79C2- | SGSGSRYDELNARLLILLAELAAERADIAAERTGDPRVRELARELKRLAQEAAEEVKRDPSSWS |
| H62A | VLHALWLIVVAIEAAVRALEAAERTGDPEVRELARELVRLAVEAAEEVQRNPSSWDVEMALLLI |
| | VFAIEAAVRALEAAERTGDPEVRELARELVRLAVEAAEEVQRNPSSKEVDMALLLILIAILEAV |
| | LSLLRAERSGDPEKREKARERVREAVERAEEVQRDPSGW (SEQ ID NO: 42) |
| tj79C2- | SGSGSREDELLARYLILLAELAAEEADIAAERTGDPRVRELARELKRLAQEAAEEVKRDPSSSD |
| H65A | VNHALHLIVLAILIAVEALEAAERTGDPEVRELARELVRLAVEAAEEVQRNPSSSDVLKALILI |
| | LLAIIQAVKALEAAERTGDPEVRELARELVRLAVEAAEEVQRNPSSKEVTMALLLILIAILEAV |
| | LSLLRAERSGDPEKREKARERVREAVERAEEVQRDPSGW (SEQ ID NO: 43) |
| tj79C2- | SGSGSREDELFARYLILLAELAAERADIAAERTGDPRVRELARELKRLAQEAAEEVKRDPSSSD |
| H65B | VNLALHLIVLAILAAVEALEAAERTGDPEVRELARELVRLAVEAAEEVQRNPSSSDVLKALMLI |
| | VLAILQAVQALEAAERTGDPEVRELARELVRLAVEAAEEVQRNPSSKEVYMALLLILIAILEAV |
| | LSLLRAERSGDPEKREKARERVREAVERAEEVQRDPSGW (SEQ ID NO: 44) |
| tj79C2- | SGSGSREDELIARYLILLAELAAERADIAAERTGDPRVRELARELKRLAQEAAEEVKRDPSSSD |
| H65C | VFMALHLIVLAIQIAVKALEAAERTGDPEVRELARELVRLAVEAAEEVQRNPSSSDVFLALLLI |
| | VTAIMLAVEALEAAERTGDPEVRELARELVRLAVEAAEEVQRNPSSKEVLMALLLILIAILEAV |
| | LSLLRAERSGDPEKREKARERVREAVERAEEVQRDPSGW (SEQ ID NO: 45) |
| tj79C2- | SGSGSREDELQARLLILLIELAAERADIAAERTGDPRVRELARELKRLAQEAAEEVKRDPSSSD |
| H65D | VWLALHLIVIAILIAVEALEAAERTGDPEVRELARELVRLAVEAAEEVQRNPSSSDVLKALMLI |
| | LIAILLLLEALEAAERTGDPEVRELARELVRLAVEAAEEVQRNPSSKEVQMALLLILIAILEAI |
| | LSLLRAERSGDPEKREKARERVREAVERAEEVQRDPSGW (SEQ ID NO: 46) |
| tj79C2- | SGSGSREDELWARLLILLIELAAERADIAAERTGDPRVRELARELKRLAQEAAEEVKRDPSSS |
| H65E | DVLLALHLIVIAILIAVEALEAAERTGDPEVRELARELVRLAVEAAEEVQRNPSSSDVLIALM |
| | LILIAILLLLEALEAAERTGDPEVRELARELVRLAVEAAEEVQRNPSSKEVQMALLLILIAIL |
| | EAILSLLRAERSGDPEKREKARERVREAVERAEEVQRDPSGW (SEQ ID NO: 47) |
| tj79C2- | SGSGSREDELQARLQLLAAELAAEEADIAAERTGDPRVRELARELKRLAQEAAEEVKRDPSSS |
| H69A | DVNAALVLITHAIMLAVEALKAAERTGDPEVRELARELVRLAVEAAEEVQRNPSSSDVNKALF |
| | LIMMAIITAVTALEAAERTGDPEVRELARELVRLAVEAAEEVQRNPSSKEVDMALLLILIAIL |
| | EAVLSLLRAERSGDPEKREKARERVREAVERAEEVQRDPSGW (SEQ ID NO: 48) |
| tj79C2- | SGSGSREDELHARYLILSAELAAERADIAAERTGDPRVRELARELKRLAQEAAEEVKRDPSSS |
| H69B | DVNKALLLITHAILIAVAALEAAERTGDPEVRELARELVRLAVEAAEEVQRNPSSSDVNRALF |
| | LIMMAIMMAVKALEAAERTGDPEVRELARELVRLAVEAAEEVQRNPSSKEVDMALLLILIAIL |
| | EAVLSLLRAERSGDPEKREKARERVREAVERAEEVQRDPSGW (SEQ ID NO: 49) |
| tj79C2- | SGSGSREDELIARYQILLAELAAERADIAAERTGDPRVRELARELKRLAQEAAEEVKRDPSSS |
| H121A | DVNMALLLILLMIELAVRALEAAERTGDPEVRELARELVRLAVEAAEEVQRNPSSSDVWQALH |
| | LIMLAIWAAVAALEAAERTGDPEVRELARELVRLAVEAAEEVQRNPSSKEVYMALLLILIAIL |
| | EAVLSLLRAERSGDPEKREKARERVREAVERAEEVQRDPSGW (SEQ ID NO: 50) |
| tj79C2- | SGSGSREDELLARLLLLLAELAAERADIAAERTGDPRVRELARELKRLAQEAAEEVKRDPSSS |
| H121B | DVLMALLLIIMAIEAAVEALEAAERTGDPEVRELARELVRLAVEAAEEVQRNPSSSDVWWALH |
| | LILFAIQLAVRALEAAERTGDPEVRELARELVRLAVEAAEEVQRNPSSKEVDMALLLILIAIL |
| | EAVLSLLRAERSGDPEKREKARERVREAVERAEEVQRDPSGW (SEQ ID NO: 51) |
| tj79C2- | SGSGSREDELLARLLILLAELAAERADIAAERTGDPRVRELARELKRLAQEAAEEVKRDPSSS |
| H121C | DVLMLLLLIVLAIQAAVEALEAAERTGDPEVRELARELVRLAVEAAEEVQRNPSSSDVEMALH |
| | LILFAIQLAVQALEAAERTGDPEVRELARELVRLAVEAAEELQRNPSSKEVQMALLLILIAIL |
| | EAVLSLLRAERSGDPEKREKARERVREAVERAEEVQRDPSGW (SEQ ID NO: 52) |
| tj79C2- | SGSGSREDELWARYTLLLAELAAERADIAAERTGDPRVRELARELKRLAQEAAEEVKRDPSSS |
| H125A | DVYLALILIVMAIYMAVLALEAAERTGDPEVRELARELVRLAVEAAEEVQRNPSSSDVNKALL |
| | LIVHAIMAAVAALEAAERTGDPEVRELARELVRLAVEAAEEVQRNPSSKEVDMALLLIQIAIL |
| | EAVLSLLRAERSGDPEKREKARERVREAVERAEEVQRDPSGW (SEQ ID NO: 53) |
| tj79C2- | SGSGSREDELRARLLILLAELAAETLDIIAERTGDPRVRELARELIRLLQEAAEEVKRDPSSS |
| H132A | DVNEALKLIVEAIFMAIFILLFAEQTGDPEVRELARELVRLAVEAAEEVQRNPSSSDVNEALK |
| | LIIQAIILAVHALLAAEKTGDPEVRELARELVRLAVEAAEEVQRNPSSKEVDMALLLILIAIL |
| | EAVLSLLRAQRSGDPEKREKARERVREAVERAEEVQRDPSGW (SEQ ID NO: 54) |
| D13_H11A | SGSGSEEKEWQAMTLHLIFAIKILKEMLEEDPNDEEIKKILELLWQISKTQQDDPHNEEQKEW |
| | LMLLLLIIMALRLVQEMLKEDPNDEEIEKILELLKQILRTLREDPHNEEQKEWLILLLIIILL |
| | LDSVQEMLKEDPNDEEIEKILELLKQILRTLREDPHNEEQKQWLIQLINALILLLFIQEKLKE |
| | DPNDEEAEEELERLKEQLRRLRE (SEQ ID NO: 55) |
| D13_H11B | SGSGSEEKEWQAMTAHLIYAIKILKEMLEEDPNDEEIKKILELLWQISKTQQDDPHNEEQKEW |
| | LILLLIIILALRLVQEMLKEDPNDEEIEKILELLKQILRTLREDPHNEEQKEWLSLLFIIIAL |
| | LDSVQEMLKEDPNDEEIEKILELLKQILRTLREDPHNEEQKQWLIQLINALILLLFIQEKLKE |
| | DPNDEEAEEELERLKEQLRRLRE (SEQ ID NO: 56) |
| D13_H11C | SGSGSEEKEWAIMTAHLIYAIKVLKEMLEEDPNDEEIKKILELLWQISKTQQDDPHNEEQKEW |
| | LMLLLGIILALRLVQEMLKEDPNDEEIEKILELLKQILRTLREDPHNEEQKEWLALLVIIILL |
| | LDSVQEMLKEDPNDEEIEKILELLKQILRTLREDPHNEEQKQWLIQLINALILLLFIQEKLKE |
| | DPNDEEAEEELERLKEQLRRLRE (SEQ ID NO: 57) |
| D13_H60A | SGSGSEEKEWMLLTAMLIMAIKTLKEMLEEDPNDEEIKKILELLWQISKTQQDDPHNEEQKEW |
| | LHLLLIIIMALRLVQEMLKEDPNDEEIEKILELLKQILRTLREDPHNEEQKEWLILLFIIIAL |
| | LDSVQEMLKEDPNDEEIEKILELLKQILRTLREDPHNEEQKQWLIQLINALILLLFIQEKLKE |
| | DPNDEEAEEELERLKEQLRRLRE (SEQ ID NO: 58) |
| D13_H60B | SGSGSEEKEWQLLTWLLIMIIKTLKEMLEEDPNDEEIKKILELLWQISKTQQDDPHNEEQKEW |
| | LHLLAAIILALRLVQEMLKEDPNDEEIEKILELLKQILRTLREDPHNEEQKAWLLLLVIIIAL |
| | LDSVQEMLKEDPNDEEIEKILELLKQILRTLREDPHNEEQKQWLIQLINALILLLFIQEKLKE |
| | DPNDEEAEEELERLKEQLRRLRE (SEQ ID NO: 59) |
| JIa | SGSSEELARESAEAAWRLAQAGTRAALAAIRGDLKELAEALIELARAVQELARVAKEYGNDEL |
| | AKTAALLAAHVALLAILVLIRAIKEGDDEVRELAKTAIKLASTAAKIVLDALPTAEEVRQITL |
| | LAKLAEEAADKKNEDSALAVGIAAAAVLAALLALEAAQKAGIEEAEKGARLLLKLAMDAARKK |
| | NPEEALAVLNAALDVSIALRLLQSAKRAGSEETRKLAEEMLRQALERARK (SEQ ID NO: |
| | 60) |
| JIc | SGSSEELARESAEAAWRLAQAATRLVLALIRGDLKEVAEALIELARAVQELARVAKEYGNDEL |
| | AKTAAWLAAHVAMLAIYVLIRAIKEGDDEVRELAKTAIKLASTAAKIVLDALPTAEEVRQITL |
| | LAKLAEEAADKKNEDSALAVGIAAIAVLIALLALEAAQKAGIEEAEKGARLLLKLAMDAARKK |
| | NPEEALAVLNAALDVSIALRLLQSAKRAGSEETRKLAEEMLRQALERARK (SEQ ID NO: |
| | 61) |
| JId | SGSSEELARESAEAAWRLAQAATRLVLAVIRGDLKEIAEALIELARAVQELARVAKEYGNDEL |
| | AKTAAFLAAHVALLAIIVLIRAIKEGDDEVRELAKTAIKLASTAAKIVLDALPTAEEVRQITL |
| | LAKLAEEAADKKNEDSALAVGIAAAAVLLALLALEAAQKAGIEEAEKGARLLLKLAMDAARKK |
| | NPEEALAVLNAALDVSIALRLLQSAKRAGSEETRKLAEEMLRQALERARK (SEQ ID NO: |
| | 62) |
| JIe | SGSSEELARESAEAAWRLAQAATRIMLAVIRGDLKEIAEALIELARAVQELARVAKEYGNDEL |
| | AKTAAFLAAHVALLAIIVLIRAIKEGDDEVRELAKTAIKLASTAAKIVLDALPTAEEVRQITL |
| | LAKLAEEAADKKNEDSALAVGIAAMAVLIALLALEAAQKAGIEEAEKGARLLLKLAMDAARKK |
| | NPEEALAVFNAALDVSIALRLLQSAKRAGSEETRKLAEEMLRQALERARK (SEQ ID NO: |
| | 63) |
| D_3_114_2 | SGSGSSDEEFKWLATWAKMAITAAERAAGTDPELQEMVALIKKELEQAERTFRNGDKSEAWR |
| | QLVFVAIAAAAVLAVALAANAAGTDPELQEMVDRILKQLKEAIRTFQNGDQEEAETQLFFVL |
| | LAAAAVAVVALALIWAGTDPELQEMVKQILEELKQAIETFARGDKEKALTQLLFVLWAAHAV |
| | LWIAIAANTAGTDPRLQQQVKEILEKLKEAIETFQKGDEEQAFRQLAEVLAEAVLVALRAMK |
| | TN (SEQ ID NO: 64) |
| D_3_114_3 | SGSGSSDELFKWLATEAKMAITAAERLAGTDPELQEMVALIKKELEQAERTERNGDKSEAMR |
| | QLLFVLMAANAVAAVAAAANMAGTDPELQEMVDRILKQLKEAIRTFQNGDQEEAMTQLAFVL |
| | LAAAAVFVVAWALILAGTDPELQEMVKQILEELKQAIETFARGDKEKALTQLLFVLWAAHAV |
| | AWIALAALLAGTDPRLLQQVKEILEKLKEAIETFQKGDEEQAFRQLAEVLAEAILVAIRAAK |
| | TN (SEQ ID NO: 65) |
| D_3_114_4 | SGSGSSDEEFKFLATMAKMAITAAERAAGTDPELQEMVALIKKELEQAERTERNGDKSEAQR |
| | QLWFVWVAALAVAAVALAANAAGTDPELQEMVDRILKQLKEAIRTFQNGDQEEAETQLRFVL |
| | MAAAAVAVVALARLWAGTDPELQEMVKQILEELKQAIETFARGDKEKALTQLLFVLWAAHAV |
| | LWIAIAANLAGTDPRLQQQVKEILEKLKEAIETFQKGDEEQAFRQLAEVLAEAVLVALRARK |
| | TN (SEQ ID NO: 66) |
| D_3_114_5 | SGSGSSDFEFKMLATWAKMAITAAERAAGTDPELQEMVALIKKELEQAERTFRNGDKSEAMR |
| | QLEFVLTAANAVLAVALAANMAGTDPELQEMVDRILKQLKEAIRTFQNGDQEEAEDQLLFVL |
| | AAATAVAVVAWAKILAGTDPELQEMVKQILEELKQAIETWARGDKEKAATQLLFVMWAAHAV |
| | AAIALAANVAGTDPRLQQQVKEILEKLKEAIETFQKGDEEQAFRQLAEVLAEAMLVAIRALL |
| | TN (SEQ ID NO: 67) |

TABLE 4
Small angle X-ray scattering (SAXS) analysis of radii of gyration (R_{G}).

| | R_{G} predicted from design | R_{G} calculated from Guinier |
|---|---|---|
| Protein name | model (Å) | analysis of SAXS data (Å) |
| SP1 | 21.9 | 27.5 |
| SP2 | 21.8 | 23.4 |
| SP3 | 21.2 | 24.4 |
| SP3x | 21.4 | 24.4 |
| JA | 20.9 | 23.3 |
| JB | 21.9 | 24.0 |
| JC | 21.8 | 26.0 |
| JD | 22.3 | 28.2 |
| JE | 21.6 | 32.1 |
| JF | 21.6 | 32.6 |
| JG | 21.6 | 28.8 |
| JH | 21.5 | 26.7 |
| D2T | 22.4 | 25.5 |
| tj79C2-H62A | 22.2 | 27.4 |
| tj79C2-H65B | 22.4 | 26.3 |
| tj79C2-H69A | 22.6 | 26.3 |
| tj79C2-H121A | 22.4 | 26.0 |
| tj79C2-H121B | 22.5 | 25.3 |
| tj79C2-H121C | 22.4 | 25.4 |
| D13_H60A | 22.7 | 25.0 |

R_{G} values were calculated from SAXS profiles shown in Supplementary FIG. 2 using Guinier Analysis in the program PRIMUS, part of the ATSAS software package (Manalastas-Cantos et al. 2021). Expected R_{G} values based on apo-states of design models and data fitting were calculated using the FoXS server (Schneidman-Duhovny et al. 2013, 2016). The systematic underprediction of R_{G} values (including for proteins with solved X-ray crystal structures) is likely the result of SAXS detection of dense hydration shells around the highly charged SP proteins that was not accounted for in design models (Kim et al. 2016; Svergun et al. 1998).

TABLE 5
ZnPPaM CD signal intensities in SP proteins compared
to organic solvent controls.

| | Absorbance | | Δε/ε |
|---|---|---|---|
| | Maximum | CD minimum or | |
| Sample | (ε, λ) | maximum (Δε, λ) | Ratio |
| ZnPPaM in | 78,000 M⁻¹cm⁻¹, | −16.2 M⁻¹cm⁻¹, 657 nm | −2.1 × 10⁻⁴ |
| DEF | 658 nm | | |
| ZnPPaM in | 76,000 M⁻¹cm⁻¹, | −17.8 M⁻¹cm⁻¹, 663 nm | −2.3 × 10⁻⁴ |
| DMSO | 660 nm | | |
| ZnPPaM in | 71,000 M⁻¹cm⁻¹, | −14.7 M⁻¹cm⁻¹, 659 nm | −2.1 × 10⁻⁴ |
| MeOH | 657 nm | | |
| SP1-ZnPPaM | 49,000 M⁻¹cm⁻¹, | −10.4 M⁻¹cm⁻¹, 661 nm | −2.1 × 10⁻⁴ |
| | 667 nm | 4.0 M⁻¹cm⁻¹, 684 nm | 0.8 × 10⁻⁴ |
| SP2-ZnPPaM | 38,200 M⁻¹cm⁻¹, | −16.5 M⁻¹cm⁻¹, 674 nm | −4.3 × 10⁻⁴ |
| | 668 nm | 12.7 M⁻¹cm⁻¹, 698 nm | 3.3 × 10⁻⁴ |
| SP3-ZnPPaM | 39,000 M⁻¹cm⁻¹, | −16.7 M⁻¹cm⁻¹, 664 nm | −4.3 × 10⁻⁴ |
| | 666 nm | 16.9 M⁻¹cm⁻¹, 694 nm | 4.3 × 10⁻⁴ |

The Δε/ε ratio gives an indication of the CD signal intensity relative to absorbance. In organic solvents, ZnPPaM has a non-conservative negative CD transition in the Q_{y} region with Δε/ε ratio of −2.1 × 10⁻⁴ to −2.3 × 10⁻⁴. In the weakly coupled ZnPPaM dimer of SP1, the transition is similarly asymmetric with a comparable As/E ratio, but a smaller positive peak is also present at 684 nm. In contrast, the conservative Cotton effects of ZnPPaM bound to either SP2 or SP3 have close to double the Δε/ε ratio, consistent with stronger excitonic coupling between the chromophores. See FIG. 2 in the main text and Supplementary FIG. 3 for spectra.

TABLE 6
Summary of X-ray crystallographic data collection and refinement statistics.

| | SP1-ZnPPaM | SP2 (apo-state) | SP2-ZnPPaM | SP3x (apo-state) |
|---|---|---|---|---|
| | (PDB ID: 7UNJ) | (PDB ID: 7UNH) | (PDB ID: 7UNI) | (PDB ID: 8EVM) |
| Well solution | 32% (w/v) PEG- | 24% (w/v) PEG- | 30% (w/v) PEG- | 2.4 M sodium |
| | 3350, 200 mM | 3350, 140 mM KCl | 3350, 100 mM | malonate dibasic |
| | lithium sulfate, | | ammonium | monohydrate, pH |
| | 100 mM BisTris | | sulfate | 7.0 |
| | pH 6.5 | | | |
| Data Collection | | | | |
| Space Group | P4₁ | P2₁ | P1 | P3₁ 2 1 |
| Unit Cell | a = b = 52.35 Å, | a = 54.13 Å, b = 76.1 Å, | a = 52.8 Å, b = 54.9 Å, | a = b = 88.732 Å, |
| Dimensions | c = 173.72 Å, | c = 63.23 Å, b = 99.06°, | c = 89.3 Å, a = 87.83°, | c = 149.484 Å, |
| | a = b = c = 90° | a = c = 90° | b = 84.06°, c = 69.45° | a = b = 90°, c = 120° |
| Resolution | 36.2-2.0 Å | 28.89-2.4 Å | 49.26-2.07 Å | 44.37-3.05 Å |
| | (2.07-2.0) | (2.46-2.4) | (2.13-2.07) | (3.16-3.05) |
| Reflections | 31367 | 19927 | 31294 | 13483 (1311) |
| Completeness (%) | 99.8 (99.5) | 98.2 (98.2) | 92.3 (60.2) | 99.42 (96.27) |
| Redundancy | 13.0 (13.2) | 48.7 (49.7) | 3.5 (3.4) | 10.8 (10.7) |
| I/s(I) | 32.4 (5.4) | 50.1 (22.7) | 7.5 (0.9) | 11.54 (0.62) |
| R_{merge} | 0.066 (0.429) | 0.082 (0.213) | 0.067 (1.297) | 0.1943 (5.037) |
| Rₚᵢₘ | 0.019 (0.121) | 0.012 (0.030) | 0.043 (0.834) | 0.0612 (1.595) |
| CC1/2 | 0.999 (0.967) | 1.01 (0.84) | 0.98 (0.97) | 0.999 (0.248) |
| Refinement | | | | |
| Resolution | 36.2-2.0 Å | 28.89-2.4 Å | 49.26-2.5 Å | 44.37-3.05 Å |
| R_{work}/R_{free} | 0.196 (0.219)/ | 0.209 (0.223)/ | 0.201 (0.258)/ | 0.2509 (0.3917)/ |
| | 0.237 (0.291) | 0.263 (0.322) | 0.251 (0.321) | 0.3017 (0.4188) |
| Rmsd bonds | 0.006 Å | 0.002 Å | 0.007 Å | 0.003 Å |
| Rmsd angles | 0.76° | 0.37° | 0.86° | 0.55° |
| Wilson B-factor | 38.31 | 22.70 | 52.90 | 112.91 |
| Number of | 3203 | 3656 | 6684 | 3539 |
| protein atoms | | | | |
| Number of ligand | 107 | 8 | 206 | 0 |
| atoms | | | | |
| Number of water | 65 | 189 | 9 | 0 |
| atoms | | | | |
| Ramachandran | 99.54/0.23/0.23 | 99.38/0.62/0.0 | 97.7/2.09/0.21 | 98.08/1.92/0.0 |
| Distribution | | | | |
| (Favored %/ | | | | |
| Allowed %/ | | | | |
| Outlier %) | | | | |

### SUPPLEMENTAL REFERENCES

Adolphs, Julia, and Thomas Renger. 2006. “How Proteins Trigger Excitation Energy Transfer in the FMO Complex of Green Sulfur Bacteria.” Biophysical Journal 91 (8): 2778-97.
Barros, Emilia P., Jamie M. Schiffer, Anastassia Vorobieva, Jiayi Dou, David Baker, and Rommie E. Amaro. 2019. “Improving the Efficiency of Ligand-Binding Protein Design with Molecular Dynamics Simulations.” Journal of Chemical Theory and Computation 15 (10): 5703-15.
Caram, Justin R., Sandra Doria, Dorthe M. Eisele, Francesca S. Freyria, Timothy S. Sinclair, Patrick Rebentrost, Seth Lloyd, and Moungi G. Bawendi. 2016. “Room-Temperature Micron-Scale Exciton Migration in a Stabilized Emissive Molecular Aggregate.” Nano Letters 16 (11): 6808-15.
Curutchet, Carles, Jacob Kongsted, Aurora Muñoz-Losa, Hoda Hossein-Nejad, Gregory D. Scholes, and Benedetta Mennucci. 2011. “Photosynthetic Light-Harvesting Is Tuned by the Heterogeneous Polarizable Environment of the Protein.” Journal of the American Chemical Societyl 33 (9): 3078-84.
Dyer, Kevin N., Michal Hammel, Robert P. Rambo, Susan E. Tsutakawa, Ivan Rodic, Scott Classen, John A. Tainer, and Greg L. Hura. 2014. “High-Throughput SAXS for the Characterization of Biomolecules in Solution: A Practical Approach.” Methods in Molecular Biology 1091: 245-58.
Georgakopoulou, Sofia, Raoul N. Frese, Evelyn Johnson, Corline Koolhaas, Richard J. Cogdell, Rienk van Grondelle, and Gert van der Zwan. 2002. “Absorption and CD Spectroscopy and Modeling of Various LH2 Complexes from Purple Bacteria.” Biophysical Journal 82 (4): 2184-97.
Huang, Xia, Cvetelin Vasilev, and C. Neil Hunter. 2020. “Excitation Energy Transfer between Monomolecular Layers of Light Harvesting LH2 and LH1-Reaction Centre Complexes Printed on a Glass Substrate.” Lab on a Chip 20 (14): 2529-38.
Kim, Henry S., Anne Martel, Eric Girard, Martine Moulin, Michael Härtlein, Dominique Madern, Martin Blackledge, Bruno Franzetti, and Frank Gabel. 2016. “SAXS/SANS on Supercharged Proteins Reveals Residue-Specific Modifications of the Hydration Shell.” Biophysical Journal 110 (10): 2185-94.
Manalastas-Cantos, Karen, Petr V. Konarev, Nelly R. Hajizadeh, Alexey G. Kikhney, Maxim V. Petoukhov, Dmitry S. Molodenskiy, Alejandro Panjkovich, et al. 2021. “ATSAS 3.0: Expanded Functionality and New Tools for Small-Angle Scattering Data Analysis.” Journal of Applied Crystallography 54 (Pt 1): 343-55.
Punjani, Ali, John L. Rubinstein, David J. Fleet, and Marcus A. Brubaker. 2017. “cryoSPARC: Algorithms for Rapid Unsupervised Cryo-EM Structure Determination.” Nature Methods 14 (3): 290-96.
Renger, Thomas, and Frank Muh. 2012. “Theory of Excitonic Couplings in Dielectric Media: Foundation of Poisson-TrEsp Method and Application to Photosystem I Trimers.” Photosynthesis Research 111 (1-2): 47-52.
Schneidman-Duhovny, Dina, Michal Hammel, John A. Tainer, and Andrej Sali. 2013. “Accurate SAXS Profile Computation and Its Assessment by Contrast Variation Experiments.” Biophysical Journal 105 (4): 962-74.
—. 2016. “FoXS, FoXSDock and MultiFoXS: Single-State and Multi-State Structural Modeling of Proteins and Their Complexes Based on SAXS Profiles.” Nucleic Acids Research 44 (W1): W424-29.

Seibt, Joachim, Dominik Lindorfer, and Thomas Renger. 2022. “Signatures of Intramolecular Vibrational and Vibronic Qx-Qycoupling Effects in Absorption and CD Spectra of Chlorophyll Dimers.” Photosynthesis Research, August. doi.org/10.1007/s11120-022-00946-3.
Svergun, D. I., S. Richard, M. H. Koch, Z. Sayers, S. Kuprin, and G. Zaccai. 1998. “Protein Hydration in Solution: Experimental Observation by X-Ray and Neutron Scattering.” Proceedings of the National Academy of Sciences of the United States of America 95 (5): 2267-72.
Weber, G., and F. W. J. Teale. 1957. “Determination of the Absolute Quantum Yield of Fluorescent Solutions.” Transactions of the Faraday Society 53 (0): 646-55.
Williams, Christopher J., Jeffrey J. Headd, Nigel W. Moriarty, Michael G. Prisant, Lizbeth L. Videau, Lindsay N. Deis, Vishal Verma, et al. 2018. “MolProbity: More and Better Reference Data for Improved All-Atom Structure Validation.” Protein Science: A Publication of the Protein Society 27 (1): 293-315.

We claim:

1. A polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-17, wherein the polypeptide binds to a chlorophyll (Chl) dimer.

2. The polypeptide of claim 1, comprising an amino acid sequence at least 75% identical to the amino acid sequence selected from the group consisting of SEQ ID NO:1-17.

3. The polypeptide of claim 1, comprising an amino acid sequence at least 75% identical to the amino acid sequence selected from the group consisting of SEQ ID NO:1-3 and 17.

4. The polypeptide of claim 1, comprising an amino acid sequence at least 85% identical to the amino acid sequence selected from the group consisting of SEQ ID NO:1-3 and 17.

5. The polypeptide of claim 3, wherein at least 1, 2, 3, 4, 5, 6, 7, 8, 9, 10, 11, 12, 13, 14, or all Chl-contacting residues are identical, or conservatively substituted, relative to the reference sequence.

6. The polypeptide of claim 3, wherein at least 1, 2, 3, 4, 5, 6, 7, 8, 9, 10, 11, 12, 13, 14, or all Chl-contacting residues are identical relative to the reference sequence.

7. The polypeptide of claim 3, wherein at least 1, 2, 3, 4, 5, 6, 7, 8, 9, 10, or all identified protein-protein residues are identical (not substituted), or conservatively substituted, relative to the reference sequence.

8. The polypeptide of claim 3, wherein at least 1, 2, 3, 4, 5, 6, 7, 8, 9, 10, 11, 12, 13, 14, or all identified protein-protein interface residues are identical, relative to the reference sequence.

9. The polypeptide of claim 3, wherein all Chl-contacting residues and all identified protein-protein are identical, relative to the reference sequence.

10. A fusion protein, comprising:
(a) the polypeptide of claim 1; and
(b) one or more functional domains at the N-terminus and/or at the C-terminus of the polypeptide.

11. A nucleic acid encoding the polypeptide of claim 1.

12. An expression vector comprising the nucleic acid of claim 11 operatively linked to a suitable control element, including but not limited to a promoter.

13. A host cell comprising the expression vector of claim 12.

14. A homodimer of the polypeptide of claim 1, further comprising a Chl dimer bound to the homodimer.

15. A scaffold comprising a plurality of the homodimers of claim 14.

16. The scaffold of claim 15, wherein the scaffold comprise
(a) a plurality of homodimers of a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:17; and
(b) a plurality of homotrimers of a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:18;
wherein the plurality of homodimers and the plurality of homotrimers non-covalently interact to form the scaffold.

17. The scaffold of claim 15, further comprising a plurality of Chl dimers bound to the plurality of homodimers.

18. A composition, comprising one or more polypeptide of claim 1 covalently linked to an electrode. Such proteins may include light-harvesting and electron-transfer domains that participate in photon absorption and charge separation. This would allow generation of electrical power upon illumination of the electrode-coupled protein complexes.

19. A method comprising use of the scaffold of claim 15 for any suitable purpose, including but not limited to as a synthetic photosystem for new energy conversion technologies, photodynamic therapy, redox- or light-responsive biosensing, fluorescent reporters, optogenetics, light-gated enzymes, photoenzymes, photoprotection in biological systems, nitrogen fixation, carbon sequestration, enhanced crop yields for food or bioenergy production, or electrical-to-fuel energy transduction for energy storage.


SEQUENCE LISTING (67 sequences)

SEQ ID NO: 1 - protein, 223 aa, synthetic construct
  VARIANT 1..2: note = optionally absent
GGDSRELIARYQILLAELAAIRADIAAERTGDPYVRKLARELKRLAQEAAEEVKRDPSSSDVNMALLLILLMIELAVRALEAAERTGDPEVRELAAELVWLAVEAAEEVQRNPSSSDVWLALHLIMLAIWAAVAALEAAERTGDPEVRELARELVRLAVEAAEEVQRNPSSKEVYMALLLILIAILEAVLSLLRAERSGDPEKREKARERVREAVERAEEVQR

SEQ ID NO: 2 - protein, 250 aa, synthetic construct
  VARIANT 1..6: note = optionally absent
  VARIANT 33: note = optionally absent
  VARIANT 89..100: note = optionally absent
  VARIANT 111..115: note = optionally absent
  VARIANT 151..169: note = optionally absent
  VARIANT 197..199: note = optionally absent
SGSGSSDEEFKFLATEAKMLITAAERLAGTDPELQEMVALIKKELEQAERTFRNGDKSEAQRQLEFVLTAARAVMNVAAAANAAGTDPELIEMVLRILKQLKEAIRTFQNGDQEEAETQLRFVLRAAIAVAVVAAALVLAGTDPELQEMVKQILEELKQAIETFARGDKEKALTQLLFVAWAAHAVAMIAAAANLAGTDPRLQQQVKEILEKLKEAIETFQKGDEEQAFRQLAEVLAEAALVALRAALTN

SEQ ID NO: 3 - protein, 239 aa, synthetic construct
  VARIANT 1..4: note = optionally absent
SGSSEELARESAEAAWRLAQAGTRLMLAWIRGDLKEIAEALIELARAVQELARVAKEYGNDELAKTAALLAAHVAMLAILVLIRAIKEGDDEVRELAKTAIKLASTAAKIVLDALPTAEEVRQITLLAKLAEEAADKKNEDSALAVGIAAAAVLLALLALEAAQKAGIEEAEKGARLLLKLAMDAARKKNPEEALAVFNAALDVSIALKLLQSAKRAGSEETRKLAEEMLRQALERARK

SEQ ID NO: 4 - protein, 231 aa, synthetic construct
  VARIANT 1..5: note = optionally absent
SGSGSRYDELNARLLILLAELAAERADIAAERTGDPRVRELARELKRLAQEAAEEVKRDPSSWSVLHALWLIVVAIEAAVRALEAAERTGDPEVRELARELVRLAVEAAEEVQRNPSSWDVFMALLLIVFAIEAAVRALEAAERTGDPEVRELARELVRLAVEAAEEVQRNPSSKEVDMALLLILIAILEAVLSLLRAERSGDPEKREKARERVREAVERAEEVQRDPSGW

SEQ ID NO: 5 - protein, 231 aa, synthetic construct
  VARIANT 1..5: note = optionally absent
SGSGSREDELLARYLILLAELAAEEADIAAERTGDPRVRELARELKRLAQEAAEEVKRDPSSSDVNHALHLIVLAILIAVEALEAAERTGDPEVRELARELVRLAVEAAEEVQRNPSSSDVLKALILILLAIIQAVKALEAAERTGDPEVRELARELVRLAVEAAEEVQRNPSSKEVTMALLLILIAILEAVLSLLRAERSGDPEKREKARERVREAVERAEEVQRDPSGW

SEQ ID NO: 6 - protein, 231 aa, synthetic construct
  VARIANT 1..5: note = optionally absent
SGSGSREDELFARYLILLAELAAERADIAAERTGDPRVRELARELKRLAQEAAEEVKRDPSSSDVNLALHLIVLAILAAVEALEAAERTGDPEVRELARELVRLAVEAAEEVQRNPSSSDVLKALMLIVLAILQAVQALEAAERTGDPEVRELARELVRLAVEAAEEVQRNPSSKEVYMALLLILIAILEAVLSLLRAERSGDPEKREKARERVREAVERAEEVQRDPSGW

SEQ ID NO: 7 - protein, 231 aa, synthetic construct
  VARIANT 1..5: note = optionally absent
SGSGSREDELIARYLILLAELAAERADIAAERTGDPRVRELARELKRLAQEAAEEVKRDPSSSDVFMALHLIVLAIQIAVKALEAAERTGDPEVRELARELVRLAVEAAEEVQRNPSSSDVFLALLLIVTAIMLAVEALEAAERTGDPEVRELARELVRLAVEAAEEVQRNPSSKEVLMALLLILIAILEAVLSLLRAERSGDPEKREKARERVREAVERAEEVQRDPSGW

SEQ ID NO: 8 - protein, 231 aa, synthetic construct
  VARIANT 1..5: note = optionally absent
SGSGSREDELQARLLILLIELAAERADIAAERTGDPRVRELARELKRLAQEAAEEVKRDPSSSDVWLALHLIVIAILIAVEALEAAERTGDPEVRELARELVRLAVEAAEEVQRNPSSSDVLKALMLILIAILLLLEALEAAERTGDPEVRELARELVRLAVEAAEEVQRNPSSKEVQMALLLILIAILEAILSLLRAERSGDPEKREKARERVREAVERAEEVQRDPSGW

SEQ ID NO: 9 - protein, 231 aa, synthetic construct
  VARIANT 1..5: note = optionally absent
SGSGSREDELWARLLILLIELAAERADIAAERTGDPRVRELARELKRLAQEAAEEVKRDPSSSDVLLALHLIVIAILIAVEALEAAERTGDPEVRELARELVRLAVEAAEEVQRNPSSSDVLIALMLILIAILLLLEALEAAERTGDPEVRELARELVRLAVEAAEEVQRNPSSKEVQMALLLILIAILEAILSLLRAERSGDPEKREKARERVREAVERAEEVQRDPSGW

SEQ ID NO: 10 - protein, 231 aa, synthetic construct
  VARIANT 1..5: note = optionally absent
SGSGSREDELQARLQLLAAELAAEEADIAAERTGDPRVRELARELKRLAQEAAEEVKRDPSSSDVNAALVLITHAIMLAVEALKAAERTGDPEVRELARELVRLAVEAAEEVQRNPSSSDVNKALFLIMMAIITAVTALEAAERTGDPEVRELARELVRLAVEAAEEVQRNPSSKEVDMALLLILIAILEAVLSLLRAERSGDPEKREKARERVREAVERAEEVQRDPSGW

SEQ ID NO: 11 - protein, 231 aa, synthetic construct
  VARIANT 1..5: note = optionally absent
SGSGSREDELHARYLILSAELAAERADIAAERTGDPRVRELARELKRLAQEAAEEVKRDPSSSDVNKALLLITHAILIAVAALEAAERTGDPEVRELARELVRLAVEAAEEVQRNPSSSDVNRALFLIMMAIMMAVKALEAAERTGDPEVRELARELVRLAVEAAEEVQRNPSSKEVDMALLLILIAILEAVLSLLRAERSGDPEKREKARERVREAVERAEEVQRDPSGW

SEQ ID NO: 12 - protein, 231 aa, synthetic construct
  VARIANT 1..5: note = optionally absent
SGSGSREDELIARYQILLAELAAERADIAAERTGDPRVRELARELKRLAQEAAEEVKRDPSSSDVNMALLLILLMIELAVRALEAAERTGDPEVRELARELVRLAVEAAEEVQRNPSSSDVWQALHLIMLAIWAAVAALEAAERTGDPEVRELARELVRLAVEAAEEVQRNPSSKEVYMALLLILIAILEAVLSLLRAERSGDPEKREKARERVREAVERAEEVQRDPSGW

SEQ ID NO: 13 - protein, 231 aa, synthetic construct
  VARIANT 1..5: note = optionally absent
SGSGSREDELLARLLLLLAELAAERADIAAERTGDPRVRELARELKRLAQEAAEEVKRDPSSSDVLMALLLIIMAIEAAVEALEAAERTGDPEVRELARELVRLAVEAAEEVQRNPSSSDVWWALHLILFAIQLAVRALEAAERTGDPEVRELARELVRLAVEAAEEVQRNPSSKEVDMALLLILIAILEAVLSLLRAERSGDPEKREKARERVREAVERAEEVQRDPSGW

SEQ ID NO: 14 - protein, 231 aa, synthetic construct
  VARIANT 1..5: note = optionally absent
SGSGSREDELLARLLILLAELAAERADIAAERTGDPRVRELARELKRLAQEAAEEVKRDPSSSDVLMLLLLIVLAIQAAVEALEAAERTGDPEVRELARELVRLAVEAAEEVQRNPSSSDVFMALHLILFAIQLAVQALEAAERTGDPEVRELARELVRLAVEAAEELQRNPSSKEVQMALLLILIAILEAVLSLLRAERSGDPEKREKARERVREAVERAEEVQRDPSGW

SEQ ID NO: 15 - protein, 231 aa, synthetic construct
  VARIANT 1..5: note = optionally absent
SGSGSREDELWARYTLLLAELAAERADIAAERTGDPRVRELARELKRLAQEAAEEVKRDPSSSDVYLALILIVMAIYMAVLALEAAERTGDPEVRELARELVRLAVEAAEEVQRNPSSSDVNKALLLIVHAIMAAVAALEAAERTGDPEVRELARELVRLAVEAAEEVQRNPSSKEVDMALLLIQIAILEAVLSLLRAERSGDPEKREKARERVREAVERAEEVQRDPSGW

SEQ ID NO: 16 - protein, 231 aa, synthetic construct
  VARIANT 1..5: note = optionally absent
SGSGSREDELRARLLILLAELAAETLDIIAERTGDPRVRELARELIRLLQEAAEEVKRDPSSSDVNEALKLIVEAIFMAIFILLFAEQTGDPEVRELARELVRLAVEAAEEVQRNPSSSDVNEALKLIIQAIILAVHALLAAEKTGDPEVRELARELVRLAVEAAEEVQRNPSSKEVDMALLLILIAILEAVLSLLRAQRSGDPEKREKARERVREAVERAEEVQRDPSGW

SEQ ID NO: 17 - protein, 245 aa, synthetic construct
  VARIANT 1: note = optionally absent
  VARIANT 238..245: note = optionally absent
MDEEFKFLATEAKMLITAAERLAGTDPRLQEMVALIKKELEQAERTFRNGDKSEAQRQLEFVLTAARAVMNVAAAANAAGTDPLLKAMVDAILWRLKEAIRTFQNGDEEAETQLRFVLRAAIAVAVVAAALVLAGTDPELQEMVEQIKDLLISAFMAGEKALTQLLFVAWAAHAVAMIAAAANLAGPRLQQQVKEILEKLKEAIETFQKGDEEQAFRQLAEVLAEAALVALRAALTNLEHHHHHH

SEQ ID NO: 18 - protein, 351 aa, synthetic construct
  VARIANT 1: note = optionally absent
  VARIANT 339..351: note = optionally absent
MSTKEKARQLAEEAKETAEKVGDPELIKLAEQASQEGDSEKAKAILLAAEAARVAKEVGAPDLIRLARIAARVGASEAAKAILLAAEAARVAKEVGDPELERLALLAAVLGDSEKAKAILLAAEAARVAKEVGDPELIKLALEAAERGDSEKAKAILLAAEAARVAKEVGDPELIKLALEAARRGDSEKAKAILLAAEAARVAKEVGDPELIKLALEAARRGDSRKAEAILLAAEAARIAKEAGDPEARKKALEAARRGDRELATRILIEALLRLLKKSTAELKRATASLRAITEELKKNPSEDALVEHNRAIVEHNAIIVENNRIIAMVLEAIVRAIGSWSGLEHHHHHH

SEQ ID NO: 19 - protein, 19 aa, synthetic construct
MGHHHHHHGSGSGENLYFQ

SEQ ID NO: 20 - protein, 223 aa, synthetic construct; identical to SEQ ID NO: 1

SEQ ID NO: 21 - protein, 250 aa, synthetic construct; identical to SEQ ID NO: 2

SEQ ID NO: 22 - protein, 239 aa, synthetic construct; identical to SEQ ID NO: 3

SEQ ID NO: 23 - protein, 241 aa, synthetic construct
SGSSEELARESAEAAWRLAQASTRATLAMIRGDLKELAEALIELARAVQELARVAKEYGNDELAKTAALLAAHVAMLAIWVLIRAIKEGDDEVRELAKTAIKLASTAAKIVLDALPTAEEVRQITLLAKLAEEAADKKNEDSALAVGIAAIAVIIALWALEAAQKAGIEEAEKGARLLLKLAMDAARKKNPEEALAVLNAALDVSIALQLLQSAKRAGSEETRKLAEEMLRQALERARKKN

SEQ ID NO: 24 - protein, 216 aa, synthetic construct
SGSGSWEEEAADKAIEIAEKVFRLKKSGTSEDEIAEEVAREISEVIRTLKESGSSEEVIALALLMIILGVVMALIANGVSIREQLDISATILYEVAREQGNEEALEAIEEITRLAEEIKEEGGSILAAHILTMAGLLVAALPSEEAAEVAKEIAKAVKAAVEAEKDGKSDEAMLALIRLILAIMAALFNRSKYSLEMVRRIAKEAEQLAEDAREGK

SEQ ID NO: 25 - protein, 241 aa, synthetic construct
SGSTEELKKVLEDVRELAERAKESTDPEEALIIAILVIIRALAAVALDPSEEASTAVKIAIKIAEEVAKRVSDSELSASIALLIANLALVAAKAAGDEELSKLALRLAKLAAELAKDALRIAEERGNAWQAAIAVGVVVMVATILLALAKQLGSEEALKEALEVAEEAARLAKRQLELAERVGDPIVALAAVGHVFMVAMLLAQIARESGSEEAKERAKRVAEEAEELAERVYELMRREGK

SEQ ID NO: 26 - protein, 242 aa, synthetic construct
SGSTEKLKKVLERVRELAERAKESTSPEEALIIALLVSSLAIAAVMLDPSEEASTAVKIAIKIAEEVAKRVSDSQLSASIALSIASLALVAAEAAGDEELSKLALRLAKLAAELAKDALRIAEERGNAIDAAIAVGIVVMVAAILLQLAKLLGSEEALKEALEVAEEAARLAKRVLELAERVGDPQVALAAVGHVQMVAILLAQIARESGSEEAKERAERVARESMELAERVLELMRREGKW

SEQ ID NO: 27 - protein, 260 aa, synthetic construct
SGSSDEEYARMSAELAEEAAKEALEQAKREGDEDARRVAEELEKQAEEARRKKDFDLANLVLMAAIILLLALIILELAKKAGNEQMEASGLSLLATAALLAAAGGENSEDARKALEAALRLAERATEVLEKIAKKGETALEKAIAAGTLVMLIQIIIQIAELLSKLGDTEEAKKVLDIAIELIKRVTEILERIAKKADTDILAQAAAGHLIYLIQLLMQISILLRRLGNIDEAFKVLEEARELAERVRELLERIAKNSDK

SEQ ID NO: 28 - protein, 237 aa, synthetic construct
SGSGSSDELAALFLIISAKSQAWQAQSAAERTGDPRVRELARELKRLAQEAAEEVKRDPSSSEVNKNLQLISMAIALAVLALQLARKAGDEEARDIAREAVEVAVRAAQLILEGRDEQRAQTLFLIAHALLMAVMAARLGNSEVAELAVKVLRLAKELEKLLPSEIALFVVLMIALAALSAAMAASGGQEDYARDALRRLEEAIREAEENRSKESLEKVIEEAIEAMLQANRAQTGG

SEQ ID NO: 29 - protein, 237 aa, synthetic construct
SGSGSSDELQARFLILSAKLQAEMAQIAAERTGDPRVRELARELKRLAQEAAEEVKRDPSSSEVNSNLALISMAIALAVLALLLARKAGDEEARDIAREAVEVAVRAAELILEGRDDQRAWTLWLIAHALLMAVYAARLGNSEVAELAVKVLRLAKELEKLLPSEEALFVVSMIGLAALQAAMAAAGGQEDYARDALRRLEEAIREAEENRSKESLEKVEEEGIEAMEQALRAIAGG

SEQ ID NO: 30 - protein, 237 aa, synthetic construct
SGSGSSDELEARLLIISANLQAEMAQIAAERTGDPRVRELARELKRLAQEAAEEVKRDPSSSEVNQNLTLISMAIALAVLALLLARKAGDEEARDIAREAVEVAVRAAELILEGRDEQRAWTLFLIAHALLLAVLAARLGNSEVAELAVKVLRLAKELEKLLPSEIALFVVSMIGMAALQAAMAASGGQEDYARDALRRLEEAIREAEENRSKESLEKVMEEAIRAYEQALRALAGG

SEQ ID NO: 31 - protein, 241 aa, synthetic construct
SGSSEELAREAREAAIRLAQASTKLMEAMIRGDLKELAEALIELARAVQELARVAKEYGNDELAKLAAFLAAHIAMLAIWVLILAIREGDDEVRELAKTAIKLASTAAKIVLDALPTAEEVRIITLLAKLAEEAADKKNEDSALAVLIAAMAVLIALAALEAAQRAGIEEAEKAARLLLKLAGEAARKKNPEEAKAVYNAALLVSIALLLLQKAKRAGSEETRRLAEEMLRQALERARKKN

SEQ ID NO: 32 - protein, 182 aa, synthetic construct
SGSGSREKELKQLISNLQILLLILLSQDPNNELVRKLQEKIKEAEEQGDPRRLKLIWWILALINILQALEQEDPNNELVKRLKELIKRALEEGDPRLLQLILLILHLIWLLQALEKEDPNNELVKRLKKLIEEALKEGDPRKLWQILALLLWIAQIQKLKKQDPNNELIKRAEETLEQSLKT

SEQ ID NO: 33 - protein, 182 aa, synthetic construct
SGSGSREKELKQLISNLQILLLILLSEDPNNELVRKLQEKIKEAEEQGDPRRLKLIWWILNLIQMLQQLEQEDPNNELVKRLKELIKRALEEGDPRLLLLIHIILALIWLLQALEKEDPNNELVKRLKKLIEEALKEGDPRKLIQIALLLTWIATIQRLKKQDPNNELIKRAEETLEQSLKT

SEQ ID NO: 34 - protein, 178 aa, synthetic construct
SGSGSEEEQIRLAQLAIQIIQYQVEKNGDEQLLRLIQEITEQLQKEGDERLIAILSTMFGIIWILEQVEKNGDEELLRLIQEITEKLARQGDERLIQIGAAMLAIIIILEQVKKNGDEQLLRLIREITRKLAEEGDQRLIWILHAMMAIIMSSEELEKNGDEELLQKLRELLEKIAKE

SEQ ID NO: 35 - protein, 186 aa, synthetic construct
SGSGSEEEEIRLAQLTIQIIQYQVEKNGDEQLLRLIQEITEQLQKEGDERLIRILNAMFTIIWILEAVEKNGDEELLRLIQEITEKLARQGDERLIKILSAMAGIIWILEIVKKNGDEQLLRLIREITRKLAEEGDERLIQILANMHSIIISSEELEKNGDEELLQKLRELLEKIAKELEHHHHHH

SEQ ID NO: 36 - protein, 178 aa, synthetic construct
SGSGSEEEEIRLAQLAIQIIQYQVEKNGDEQLLRLIQEITEQLQKEGDERLIRILLAMFAIILILEHVEKNGDEELLRLIQEITEKLARQGDERLIRILIAMAGIIWILELVKKNGDEQLLRLIREITRKLAEEGDERLIQILLAMHAIIISSEQLEKNGDEELLQKLRELLEKIAKE

SEQ ID NO: 37 - protein, 180 aa, synthetic construct
SGSGSEEEEKRLIEKMSKFIWWFAQQLGDERILRAVEKLQQEIKENSPTPETFMTLINLLLSLLIWWAAQKLGDEKILEAVERLLQQIEENSPTPETHMTLAALAQSLAIWQQAQKLGDEDILEAVERLLEEIQRNSPTPSTLIKLTWLLLLLNNWQELQKQGDEEQLRSSEKAMKELKE

SEQ ID NO: 38 - protein, 180 aa, synthetic construct
SGSGSEEEEKRLIEKMSKFIWWFAQQLGDERILRAVEKLQQEIKENSPTPETYMTLGNLLLSLLIWWAAQQLGDEKILEAVERLLQQIEENSPTPETHLTLAILMSALAIWQQAQKLGDEDILEAVERLLEEIQRNSPTPSTMTKLLWLALLLNNWEELQKQGDEEQLRSSEKAMKELKE

SEQ ID NO: 39 - protein, 212 aa, synthetic construct
SGSGSEEEELKRLQTLIKYLQLLIQNAKWQNPDDELLRQVEELIEEMKKRLEENPNSQEDQALLKLLITIIALIIMILWLKSENPDDELLRQVEELIKEMIERLKENPNSQEDQALLQLLHAILGLILLIQQLKKQNPDDELLQQVEELIEEMIKRLKENPNSEKDQILLSFLALSLLSIFLIQTQKKQNPDDERLKQWEKQIKESIESSKE

SEQ ID NO: 40 - protein, 212 aa, synthetic construct
SGSGSEEEELERLKEQLKIWQKLIKLILEQDPNDEQIRRILEQAEEIIQKLQEDPNNEELQRLIQLLFNLVAIIFSIKQILELDPNDEQIRRILEQAKEIIQKLQEDPNNEELFLLAHMLFALVAIILLIKLMLEQDPNDELIREILEQAKEIIQKLQEDPNNAELAMLIALLLSLVLWIWFMKFQLEQDPNDEEIRRQLEQLEERIRRTLE

SEQ ID NO: 41 - protein, 212 aa, synthetic construct
SGSGSEEEELERLKEQLKIWQKLIKLILEQDPNDEQIRRILEQAEEIIQKLQEDPNNEELQRLIQLLFQLVLGIFIIKQILELDPNDEQIRRILEQAKEIIQKLQEDPNNEELYRLQGLLGMLVGIILLIKLMLEQDPNDELIREILEQAKEIIQKLQEDPNNQELLSLIHLLLALVLWIWFMKFQLEQDPNDEEIRRQLEQLEERIRRTLE

SEQ ID NO: 42 - protein, 231 aa, synthetic construct; identical to SEQ ID NO: 4

SEQ ID NO: 43 - protein, 231 aa, synthetic construct; identical to SEQ ID NO: 5

SEQ ID NO: 44 - protein, 231 aa, synthetic construct; identical to SEQ ID NO: 6

SEQ ID NO: 45 - protein, 231 aa, synthetic construct; identical to SEQ ID NO: 7

SEQ ID NO: 46 - protein, 231 aa, synthetic construct; identical to SEQ ID NO: 8

SEQ ID NO: 47 - protein, 231 aa, synthetic construct; identical to SEQ ID NO: 9

SEQ ID NO: 48 - protein, 231 aa, synthetic construct; identical to SEQ ID NO: 10

SEQ ID NO: 49 - protein, 231 aa, synthetic construct; identical to SEQ ID NO: 11

SEQ ID NO: 50 - protein, 231 aa, synthetic construct; identical to SEQ ID NO: 12

SEQ ID NO: 51 - protein, 231 aa, synthetic construct; identical to SEQ ID NO: 13

SEQ ID NO: 52 - protein, 231 aa, synthetic construct; identical to SEQ ID NO: 14

SEQ ID NO: 53 - protein, 231 aa, synthetic construct; identical to SEQ ID NO: 15

SEQ ID NO: 54 - protein, 231 aa, synthetic construct; identical to SEQ ID NO: 16

SEQ ID NO: 55 - protein, 212 aa, synthetic construct
SGSGSEEKEWQAMTLHLIFAIKILKEMLEEDPNDEEIKKILELLWQISKTQQDDPHNEEQKEWLMLLLLIIMALRLVQEMLKEDPNDEEIEKILELLKQILRTLREDPHNEEQKEWLILLLIIILLLDSVQEMLKEDPNDEEIEKILELLKQILRTLREDPHNEEQKQWLIQLINALILLLFIQEKLKEDPNDEEAEEELERLKEQLRRLRE

SEQ ID NO: 56 - protein, 212 aa, synthetic construct
SGSGSEEKEWQAMTAHLIYAIKILKEMLEEDPNDEEIKKILELLWQISKTQQDDPHNEEQKEWLILLLIIILALRLVQEMLKEDPNDEEIEKILELLKQILRTLREDPHNEEQKEWLSLLFIIIALLDSVQEMLKEDPNDEEIEKILELLKQILRTLREDPHNEEQKQWLIQLINALILLLFIQEKLKEDPNDEEAEEELERLKEQLRRLRE

SEQ ID NO: 57 - protein, 212 aa, synthetic construct
SGSGSEEKEWAIMTAHLIYAIKVLKEMLEEDPNDEEIKKILELLWQISKTQQDDPHNEEQKEWLMLLLGIILALRLVQEMLKEDPNDEEIEKILELLKQILRTLREDPHNEEQKEWLALLVIIILLLDSVQEMLKEDPNDEEIEKILELLKQILRTLREDPHNEEQKQWLIQLINALILLLFIQEKLKEDPNDEEAEEELERLKEQLRRLRE

SEQ ID NO: 58 - protein, 212 aa, synthetic construct
SGSGSEEKEWMLLTAMLIMAIKTLKEMLEEDPNDEEIKKILELLWQISKTQQDDPHNEEQKEWLHLLLIIIMALRLVQEMLKEDPNDEEIEKILELLKQILRTLREDPHNEEQKEWLILLFIIIALLDSVQEMLKEDPNDEEIEKILELLKQILRTLREDPHNEEQKQWLIQLINALILLLFIQEKLKEDPNDEEAEEELERLKEQLRRLRE

SEQ ID NO: 59 - protein, 212 aa, synthetic construct
SGSGSEEKEWQLLTWLLIMIIKTLKEMLEEDPNDEEIKKILELLWQISKTQQDDPHNEEQKEWLHLLAAIILALRLVQEMLKEDPNDEEIEKILELLKQILRTLREDPHNEEQKAWLLLLVIIIALLDSVQEMLKEDPNDEEIEKILELLKQILRTLREDPHNEEQKQWLIQLINALILLLFIQEKLKEDPNDEEAEEELERLKEQLRRLRE

SEQ ID NO: 60 - protein, 239 aa, synthetic construct
SGSSEELARESAEAAWRLAQAGTRAALAAIRGDLKELAEALIELARAVQELARVAKEYGNDELAKTAALLAAHVALLAILVLIRAIKEGDDEVRELAKTAIKLASTAAKIVLDALPTAEEVRQITLLAKLAEEAADKKNEDSALAVGIAAAAVLAALLALEAAQKAGIEEAEKGARLLLKLAMDAARKKNPEEALAVLNAALDVSIALRLLQSAKRAGSEETRKLAEEMLRQALERARK

SEQ ID NO: 61 - protein, 239 aa, synthetic construct
SGSSEELARESAEAAWRLAQAATRLVLALIRGDLKEVAEALIELARAVQELARVAKEYGNDELAKTAAWLAAHVAMLAIYVLIRAIKEGDDEVRELAKTAIKLASTAAKIVLDALPTAEEVRQITLLAKLAEEAADKKNEDSALAVGIAAIAVLIALLALEAAQKAGIEEAEKGARLLLKLAMDAARKKNPEEALAVLNAALDVSIALRLLQSAKRAGSEETRKLAEEMLRQALERARK

SEQ ID NO: 62 - protein, 239 aa, synthetic construct
SGSSEELARESAEAAWRLAQAATRLVLAVIRGDLKEIAEALIELARAVQELARVAKEYGNDELAKTAAFLAAHVALLAIIVLIRAIKEGDDEVRELAKTAIKLASTAAKIVLDALPTAEEVRQITLLAKLAEEAADKKNEDSALAVGIAAAAVLLALLALEAAQKAGIEEAEKGARLLLKLAMDAARKKNPEEALAVLNAALDVSIALRLLQSAKRAGSEETRKLAEEMLRQALERARK

SEQ ID NO: 63 - protein, 239 aa, synthetic construct
SGSSEELARESAEAAWRLAQAATRIMLAVIRGDLKEIAEALIELARAVQELARVAKEYGNDELAKTAAFLAAHVALLAIIVLIRAIKEGDDEVRELAKTAIKLASTAAKIVLDALPTAEEVRQITLLAKLAEEAADKKNEDSALAVGIAAMAVLIALLALEAAQKAGIEEAEKGARLLLKLAMDAARKKNPEEALAVFNAALDVSIALRLLQSAKRAGSEETRKLAEEMLRQALERARK

SEQ ID NO: 64 - protein, 250 aa, synthetic construct
SGSGSSDEEFKWLATWAKMAITAAERAAGTDPELQEMVALIKKELEQAERTFRNGDKSEAWRQLVFVAIAAAAVLAVALAANAAGTDPELQEMVDRILKQLKEAIRTFQNGDQEEAETQLFFVLLAAAAVAVVALALIWAGTDPELQEMVKQILEELKQAIETFARGDKEKALTQLLFVLWAAHAVLWIAIAANTAGTDPRLQQQVKEILEKLKEAIETFQKGDEEQAFRQLAEVLAEAVLVALRAMKTN

SEQ ID NO: 65 - protein, 250 aa, synthetic construct
SGSGSSDELFKWLATEAKMAITAAERLAGTDPELQEMVALIKKELEQAERTFRNGDKSEAMRQLLFVLMAANAVAAVAAAANMAGTDPELQEMVDRILKQLKEAIRTFQNGDQEEAMTQLAFVLLAAAAVFVVAWALILAGTDPELQEMVKQILEELKQAIETFARGDKEKALTQLLFVLWAAHAVAWIALAALLAGTDPRLLQQVKEILEKLKEAIETFQKGDEEQAFRQLAEVLAEAILVAIRAAKTN

SEQ ID NO: 66 - protein, 250 aa, synthetic construct
SGSGSSDEEFKFLATMAKMAITAAERAAGTDPELQEMVALIKKELEQAERTFRNGDKSEAQRQLWFVWVAALAVAAVALAANAAGTDPELQEMVDRILKQLKEAIRTFQNGDQEEAETQLRFVLMAAAAVAVVALARLWAGTDPELQEMVKQILEELKQAIETFARGDKEKALTQLLFVLWAAHAVLWIAIAANLAGTDPRLQQQVKEILEKLKEAIETFQKGDEEQAFRQLAEVLAEAVLVALRARKTN

SEQ ID NO: 67 - protein, 250 aa, synthetic construct
SGSGSSDFEFKMLATWAKMAITAAERAAGTDPELQEMVALIKKELEQAERTFRNGDKSEAMRQLEFVLTAANAVLAVALAANMAGTDPELQEMVDRILKQLKEAIRTFQNGDQEEAEDQLLFVLAAATAVAVVAWAKILAGTDPELQEMVKQILEELKQAIETWARGDKEKAATQLLFVMWAAHAVAAIALAANVAGTDPRLQQQVKEILEKLKEAIETFQKGDEEQAFRQLAEVLAEAMLVAIRALLTN